(19) United States
(12) Patent Application Publication
(10) Pub. No.: US 2026/0242467 A1
(43) Pub. Date: Aug. 20, 2026

(54) CHIMERIC IMMUNOCYTOKINES

(71) Applicant: DANA-FARBER CANCER INSTITUTE, INC., Boston, MA (US)

(72) Inventors: Rizwan ROMEE, Boston, MA (US); Alaa ALI, Boston, MA (US)

(73) Assignee: DANA-FARBER CANCER INSTITUTE, INC., Boston, MA (US)

(21) Appl. No.: 19/161,624

(22) PCT Filed: Mar. 1, 2024
(86) PCT No.: PCT/US2024/018070

§ 371 Date: Sep. 2, 2025

Related U.S. Application Data
(60) Provisional application No. 63/449,652, filed on Mar. 3, 2023.

(51) Int. Cl. C07K 16/28; A61K 35/00; A61K 38/00; A61K 40/15; A61K 40/31; A61K 40/42; C07K 14/54

(52) U.S. Cl. CPC C07K 16/2803; A61K 35/00; A61K 40/15; A61K 40/31; A61K 40/421; C07K 14/5443; A61K 38/00; C07K 2317/53; C07K 2319/33

(57) ABSTRACT

Disclosed are mabkines containing a target binding domain, that binds an antigen on a cancer cell, a cognate receptor or cognate ligand of the antigen on an immune cell, a hinge domain, an IL-15Rα domain, a linker, and an IL-15 domain.

### CROSS-REFERENCE TO RELATED APPLICATIONS

This application claims the benefit of priority under 35 U.S.C. § 119(e) to U.S. Provisional Application No. 63/449,652, filed Mar. 3, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 16, 2023, is named 52095_769001WO_ST.xml and is 93 KB bytes in size.

### BACKGROUND OF THE DISCLOSURE

Tumors develop a unique ecosystem during tumor development, commonly referred to as the tumor microenvironment. The tumor microenvironment shields the tumor from the immune system, in part through the recruitment of immunosuppressive cells such as regulatory T cells, macrophages, and myeloid-derived suppressor cells (MDSCs) that suppress cytotoxic T and NK cells.

One important immunosuppressive pathway that tumors take advantage of is the NKG2A/HLA-E pathway. NKG2A is expressed on cytotoxic lymphocytes, including natural killer cells and CD8 T cells. NKG2A+ cells preferentially reside in tissues, including the tumor microenvironment. The NKG2A ligand, histocompatibility leucocyte antigen E (HLA-E), is a conserved nonclassical HLA class I molecule that binds a limited peptide repertoire. HLA-E expression is commonly detected in human cancers, suggesting that blocking the NKG2A/HLA-E pathway is a promising cancer target (Borst et al., Clin. Cancer Res. 26:5549-5556 (2020)). However, NKG2A blockade as a standalone therapy has been poorly effective in mouse tumor models. Therefore, new, more effective therapeutics that target immunosuppressive pathways are needed.

### SUMMARY OF THE DISCLOSURE

A first aspect of the present disclosure is directed to a fusion protein that contains a) a target binding domain that binds an antigen on a cancer cell, or wherein the target binding domain binds a cognate receptor of the antigen, or a cognate ligand of the antigen, wherein the cognate receptor and cognate ligand are present an immune cell, b) a hinge domain, c) an IL-15Rα domain, d) a linker, and e) an IL-15 domain, wherein the hinge domain is disposed between the target binding domain and the IL-15Rα domain, and the linker is disposed between the IL-15Rα domain and the IL-15 domain. The fusion proteins are also referred to herein as mabkines.

Yet another aspect of the present disclosure is directed to a nucleic acid that encodes the mabkine.

Yet another aspect of the present disclosure is directed to a vector that contains the nucleic acid encoding the mabkine.

Another aspect of the present disclosure is directed to a genetically modified cell that contains the vector. In some embodiments, the genetically modified cell is a mammalian immune cell.

Another aspect of the present disclosure is directed to a method of making a mabkine. The method entails introducing the vector into a cell, culturing the cell in a medium under conditions where the nucleic acid encoding the mabkine is expressed, and isolating the mabkine from the cell and/or the medium.

Yet another aspect of the present disclosure is directed to a pharmaceutical composition that contains the mabkine, and a pharmaceutically acceptable carrier. A related aspect is directed to a pharmaceutical composition that contains a genetically modified immune cell that contains the mabkine and a pharmaceutically acceptable carrier.

Yet another aspect of the present disclosure is directed to a method of treating cancer. In some embodiments, the method entails administering to a subject in need thereof a pharmaceutical composition containing an effective amount of the mabkine. In some embodiments, the method entails administering to a subject in need thereof a pharmaceutical composition containing an effective amount of genetically modified immune cells that contain the nucleic acid encoding the mabkine and a pharmaceutically acceptable carrier. In some embodiments, the method entails co-administering to the subject thereof an effective number of genetically modified immune cells that contain a nucleic acid encoding a CAR.

As shown in working examples herein, the present inventors have found that NK cell-targeting mabkines enhance NK cell activation and target cell killing.

### BRIEF DESCRIPTION OF THE DRAWINGS

FIGS. 1A-1C are a set of schematics that show the design of mabkines according to one embodiment. FIG. 1A schematically illustrates the domains of three mabkines. FIG. 1B schematically illustrates the domains of a homodimerized mabkine #1. FIG. 1C illustrates the domains of a homodimerized mabkine #3.

FIGS. 2A-2B are a set of schematics that show vectors containing mabkine-encoding nucleic acids. FIG. 2A schematically illustrates the UCOE® Human 4 kb Purpo Set vector backbone. FIG. 2B schematically illustrates the methodology of digesting and ligating an mabkine-encoding nucleic acid from a donor vector to a recipient vector.

FIG. 3 is a photograph of a gel loaded with UCOE® vectors with and without an mabkine-encoding nucleic acid digested with different restriction enzymes.

FIGS. 4A-4C are a set of flow cytometry plots that show NKG2A expression in Natural Killer (NK) cell subsets. FIG. 4A is a set of flow cytometry plots that show the gating strategy for identifying CD56⁺ NK cells. FIG. 4B is a set of flow cytometry plots that show the gating strategy for identifying CD56⁺ NK cell subsets. FIG. 4C is a set of flow cytometry plots that show NKG2A expression in CD56⁺ NK cell subsets.

FIGS. 5A-5C are a set of flow cytometry plots that show the gating strategy and NKG2A expression on expanded primary NK cells and chimeric antigen receptor (CAR) expressing-NK cells. FIG. 5A is a set of flow cytometry plots that show the gating strategy for identifying untransduced and CAR-expressing CD56⁺ NK cells. FIG. 5B is a set of flow cytometry plots that show the gating strategy for identifying CD56⁺ NK cells and GFP⁺ cells, where the CAR-construct encodes GFP. FIG. 5C is a set of flow cytometry plots that show NKG2A expression in GFP⁻ and GFP⁻ NK cells.

FIG. 6 is a set of flow cytometry plots that show the gating strategy and NKG2A expression in the NK92 MI NK cell line.

FIGS. 7A-7F are a set of flow cytometry plots showing that mabkines competitively bind to NK cells in the presence of anti-NKG2A antibodies. FIG. 7A is a set of flow cytometry plots that show anti-NKG2A binding NK cells without mabkine (0 μM; negative control). FIG. 7B is a set of flow cytometry plots that show anti-NKG2A antibody binding to NK cells following treatment with 0.08 μM of mabkine. FIG. 7C is a set of flow cytometry plots that show anti-NKG2A antibody binding to NK cells following treatment with 0.2 μM of mabkine. FIG. 7D is a set of flow cytometry plots that show anti-NKG2A antibody binding to NK cells following treatment with 0.8 μM of mabkine. FIG. 7E is a set of flow cytometry plots that show anti-NKG2A antibody binding to NK cells following treatment with 2 μM of mabkine. FIG. 7F is a set of flow cytometry plots that show anti-NKG2A antibody binding to NK cells following treatment with 8 μM of mabkine.

FIG. 8 is a set of flow cytometry plots that show the gating strategy of purified NK cells into CD56^{bright} and CD56^{dim} NK cells for phosphorylated STAT5 (pSTAT5).

FIGS. 9A-9F are a set of flow cytometry plots that show expression of pSTAT5 in NK cell subsets after IL-15 stimulation. FIG. 9A is a set of flow cytometry plots that show NK cells without IL-15 (0 μM; negative control). FIG. 9B is a set of flow cytometry plots that show NK cells stimulation with 0.08 μM of IL-15. FIG. 9C is a set of flow cytometry plots that show NK cells stimulation with 0.2 μM of IL-15. FIG. 9D is a set of flow cytometry plots that show NK cells stimulation with 0.8 μM of IL-15. FIG. 9E is a set of flow cytometry plots that show NK cells stimulation with 2 μM of IL-15. FIG. 9F is a set of flow cytometry plots that show NK cells stimulation with 8 μM of IL-15.

FIGS. 10A-10F are a set of flow cytometry plots that show expression of pSTAT5 in NK cell subsets mabkine stimulation. FIG. 10A is a set of flow cytometry plots that show NK cells without mabkine (0 μM; negative control). FIG. 10B is a set of flow cytometry plots that show NK cells stimulation with 0.08 μM of mabkine. FIG. 10C is a set of flow cytometry plots that show NK cells stimulation with 0.2 μM of mabkine. FIG. 10D is a set of flow cytometry plots that show NK cells stimulation with 0.8 μM of mabkine. FIG. 10E is a set of flow cytometry plots that show NK cells stimulation with 2 μM of mabkine. FIG. 10F is a set of flow cytometry plots that show NK cells stimulation with 8 μM of mabkine.

FIGS. 11A-11B are a set of line and bar plots that show NK cell killing of parental Ramos cells and Ramos cells overexpressing an HLA-E transgene in the presence of different concentrations of IL-15 or mabkine. FIG. 11A is a line plot and a bar plot that shows the effect of IL-15 on NK cell killing of target cells. FIG. 11B is a line plot and a bar plot that shows the effect of mabkine on NK cell killing of target cells.

FIGS. 12A-12B are a set of line plots that show NK cell killing of parental Ramos cells and Ramos cells overexpressing an HLA-E transgene containing the HLA-G signal peptide (GpHLA-E) with and without IL-15 or mabkine treatment. FIG. 12A is a set of line plots that shows the effect of GpHLA-E on NK cell killing of target cells with and without IL-15 or mabkine treatment. FIG. 12B is a set of line plots that shows the effect of IL-15 or mabkine treatment on NK cell killing of target cells with and without overexpression of GpHLA-E.

FIGS. 13A-13B are a set of line plots that show CAR-NK cell killing of parental Ramos cells and Ramos cells overexpressing GpHLA-E with and without IL-15 or mabkine treatment. FIG. 13A is a set of line plots that shows the effect of GpHLA-E on CAR-NK cell killing of target cells with and without IL-15 or mabkine treatment. FIG. 13B is a set of line plots that shows the effect of IL-15 or mabkine treatment on NK cell killing of target cells with and without overexpression of GpHLA-E.

FIGS. 14A-14B are a set of line plots that show NK cell killing of parental SKBR3 cells and SKBR3 cells overexpressing GpHLA-E with and without IL-15 or mabkine treatment. FIG. 14A is a line plot that shows the effect of GpHLA-E on untransduced NK and CAR-NK cell killing of target cells. FIG. 14B is a line plot that shows the effect of mabkine treatment on untransduced NK and CAR-NK cell killing of target cells overexpressing GpHLA-E.

FIGS. 15A-15C are a set of bar plots that show NK cell proportions, numbers, and proliferation in human peripheral blood mononuclear cells (PBMCs) after mabkine or IL-15 treatment. FIG. 15A is a bar plot that shows the proportion of NK cells in human PBMCs. FIG. 15B is a bar plot that shows NK cell counts in human PBMCs. FIG. 15C is a bar plot that shows the percentages of NK cells in human PBMCs that have undergone cell proliferation.

FIGS. 16A-16C are a set of bar plots that show T cell proportions, numbers, and proliferation in human PBMCs after mabkine or IL-15 treatment. FIG. 16A is a bar plot that shows the proportion of T cells in human PBMCs. FIG. 16B is a bar plot that shows T cell counts in human PBMCs. FIG. 16C is a bar plot that shows the percentages of T cells in human PBMCs that have undergone cell proliferation.

FIGS. 17A-17D are a set of bar plots that show CD4⁺ T and CD8⁺ T cell numbers and proliferation in human PBMCs after mabkine or IL-15 treatment. FIG. 17A is a bar plot that shows CD4⁺ T cell counts in human PBMCs. FIG. 17B is a bar plot that shows the percentages of CD4⁻ T cells in human PBMCs that have undergone cell proliferation. FIG. 17C is a bar plot that shows CD8⁺ T cell counts in human PBMCs. FIG. 17D is a bar plot that shows the percentages of CD8⁺ T cells in human PBMCs that have undergone cell proliferation.

### DETAILED DESCRIPTION OF THE DISCLOSURE

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “include(s)”, “including,” “contain(s)”, “containing,” or “characterized by,” is inclusive or open-ended and does not exclude additional, unrecited elements or method steps. By contrast, the transitional phrases “consist(s) of” and “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### Mabkines

In one aspect, the disclosure provides an mabkine containing a fusion protein that contains a target binding domain that binds an antigen on a cancer cell, a cognate receptor of the antigen, or a cognate ligand of the antigen, a hinge domain, an interleukin-15 (IL-15) receptor (IL-15R) α domain, a linker and an IL-15 domain, where the cognate receptor and the cognate ligand are present on an immune cell, the hinge domain is disposed between the target binding domain and the IL-15Rα domain, and the linker is disposed between the IL-15Rα domain and the IL-15 domain. The mabkine provides increased specificity of the cytokine portion (i.e., the IL-15R binding domain, linker, and IL-15 domain) towards a particular cell type, i.e., a tumor cell or an immune cell, through the target binding domain.

### Target Binding Domain

The target binding domain specifically binds an antigen on a cancer cell, a cognate receptor of the antigen, or a cognate ligand of the antigen, where the cognate receptor and the cognate ligand are on an immune cell. The term “antigen” as used herein refers to an entity at least a portion of which is present on the surface of a cancer cell. Antigens may be proteins, peptides, peptide-protein complexes (e.g., a peptide bound to an MHC molecule), protein-carbohydrate complexes (e.g., a glycoprotein), protein-lipid complexes (e.g., a lipoprotein), protein-nucleic acid complexes (e.g., a nucleoprotein), carbohydrate, lipid, or nucleic acid.

The terms “cognate ligand” and “cognate receptor” of an antigen refer to molecules (e.g., proteins) naturally present on an immune cell to which the antigen binds. Therefore, binding of the cognate ligand or cognate receptor by the target binding domain prevents the antigen's binding its cognate receptor or cognate ligand, and prevents signal transduction in the immune cell, and positions the mabkine in close proximity to the cell surface of the immune cell, enabling binding of the mabkine's IL-15 domain to an IL-15R on the immune cell.

In some embodiments, the target binding domain is a single-chain variable antibody fragment (scFv) that includes a variable light (VL) and a variable heavy (VH) domain that may be derived from an immunoglobulin that binds the antigen. The term “derived from” as used herein when referring to protein or nucleic acid sequences refers to a sequence that originates from another, parent sequence. A sequence derived from a parent sequence may be identical, may be a portion of the parent sequence, or may have at least one variant from the parent sequence. Variants may include substitutions, insertions, or deletions. Thus, for example, an amino acid sequence derived from a parent sequence may be identical for a specific range of amino acids of the parent but does not include amino acids outside that specific region.

In some embodiments, the target binding domain binds an antigen on a cancer cell. In some embodiments, the target binding domain binds EGFR, PD-1, PD-L1, or HLA-E. In some embodiments, the mabkine targets the EGF/EGFR pathway by binding EGFR. In some embodiments, the target binding domain is a scFv that binds EGFR. In some embodiments, the target binding domain is derived from the sequence of a commercially available anti-EGFR antibody, antibody fragment, or variant thereof, for example, cetuximab (Erbitux®), panitumumab (Vectibix®), necitumumab (Portrazza®), and amivantamab (Rybrevant®), the amino acid sequences of the heavy and light chains of which are set forth in Table 1.

TABLE 1
Amino Acid Sequences of Representative anti-EGFR Antibody Fragments

```
cetuximab heavy chain (SEQ ID NO: 1)
    1 qvqlkqsgpg lvqpsqslsi tctvsgfslt nygvhwvrqs pgkglewlgv iwsggntdyn
  61 tpftsrlsin kdnsksqvff kmnslqsndt aiyycaralt yydyefaywg qgtlvtvsaa
121 stkgpsvfpl apsskstsgg taalgclvkdyfpepvtvsw nsgaltsgvh tfpavlqssg
181 lyslssvvtv pssslgtqty icnvnhkpsn tkvdkrvepk scdkthtcpp cpapellggp
241 svflfppkpk dtlmisrtpe vtcvvvdvsh edpevkfnwy vdgvevhnak tkpreeqyns
301 tyrvvsvltv lhqdwlngke ykckvsnkal papiektisk akgqprepqv ytlppsreem
361 tknqvsltcl vkgfypsdia vewesngqpe nnykttppvl dsdgsfflys kltvdksrwq
421 qgnvfscsvm healhnhytq kslslspgk
cetuximab light chain (SEQ ID NO: 2)
    1 dilltqspvi lsvspgervs fscrasqsig tnihwyqqrt ngsprlliky asesisgips
  61 rfsgsgsgtd ftlsinsves ediadyycqq nnnwpttfga gtklelkrtv aapsvfifpp
121 sdeqlksgta svvclInnfy preakvqwkv dnalqsgnsq esvteqdskd styslsstlt
181 lskadyekhk vyacevthqg lsspvtksfn rgec
panitumumab heavy chain (SEQ ID NO: 3)
    1 qvqlqesgpg lvkpsetlsl tctvsggsvs sgdyywtwir qspgkglewi ghiyysgntn
  61 ynpslksrlt isidtsktqf slklssvtaa dtaiyycvrd rvtgafdiwg qgtmvtvssa
121 stkgpsvipl apcsrstses taalgclvkd yfpepvtvsw nsgaltsgvh tfpavlqssg
181 lyslssvvtv pssnfgtqty tcnvdhkpsn tkvdktverk c
panitumumab light chain (SEQ ID NO: 4)
    1 diqmtqspss lsasvgdrvt itcqasqdis nylnwyqqkp gkapklliyd asnletgvps
  61 rfsgsgsgtd ftftisslqp ediatyfcqh fdhlplafgg gtkveikrtv aapsvfifpp
121 sdeqlksgta svvclInnfy preakvqwkv dnalqsgnsq esvteqdskd styslsstlt
181 lskadyekhk vyacevthqg lsspvtksfn rgec
necitumumab heavy chain (SEQ ID NO: 5)
    1 qvqlqesgpg lvkpsqtlsl tctvsggsis sgdyywswir qppgkglewi gyiyysgstd
  61 ynpslksrvt msvdtsknqf slkvnsvtaa dtavyycarv sifgvgtfdy wgqgtlvtvs
121 sastkgpsvl plapssksts ggtaalgclv kdyfpepvtv swnsgaltsg vhtfpavlqs
181 sglyslssvv tvpssslgtq tyicnvnhkp sntkvdkrve pkscdkthtc ppcpapellg
241 gpsvflfppk pkdtlmisrt pevtcvvvdv shedpevkfn wyvdgvevhn aktkpreegy
301 nstyrvvsvl tvlhqdwlng keykckvsnk alpapiekti skakgqprep qvytlppsre
361 emtknqvslt clvkgfypsd iavewesngq pennykttpp vldsdgsffl yskltvdksr
421 wqqgnvfscs vmhealhnhy tqkslslspg k
necitumumab heavy chain (SEQ ID NO: 6)
    1 eivmtqspat lslspgerat lscrasqsvs sylawyqqkp gqaprlliyd asnratgipa
  61 rfsgsgsgtd ftltisslep edfavyychq ygstpltfgg gtkaeikrtv aapsvfifpp
121 sdeqlksgta svvclInnfy preakvqwkv dnalqsgnsq esvteqdskd styslsstlt
181 lskadyekhk vyacevthqg lsspvtksfn rgec
```

In some embodiments, the target binding domain binds PD-1. In some embodiments, the target binding domain is derived from a commercially available anti-PD-1 antibody, antibody fragment, or derivative thereof, e.g., balstilimab, budigalimab, cadonilimab, cemiplimab (Libtayo®), cetrelimab, dostarlimab (Jemperli®), izuralimab, nivolumab (Opdivo®), pacmilimab, pembrolizumab (Keytruda®), penpulimab, peresolimab, pidilizumab, retifanlimab, rosnilimab, sintilimab, spartalizumab, tislelizumab, toripalimab, volrustomig, vudalimab, zeluvalimab, and zimberelimab, the amino acid sequences of representative heavy and light chains of which are set forth in Table 2.

TABLE 2
Amino Acid Sequence of Representative anti-PD-1 Antibody Fragments

```
balstilimab heavy chain (SEQ ID NO: 76)
    1 qvqlvesggg vvqpgrslrl scaasgftfs sygmhwvrqa pgkglewvav iwydgsnkyy
  61 adsvkgriti srdnskntly lqmnslraed tavyycasng dhwgqgtlvt vssastkgps
121 vfplapcsrs tsestaalgc lvkdyfpepv tvswnsgalt sgvhtfpavl qssglyslss
181 vvtvpssslg tktytcnvdh kpsntkvdkr veskygppcp pcpapeflyg psvflfppkp
241 kdtlmisrtp evtcvvvdvs qedpevqfnw yvdgvevhna ktkpreeqfn styrvvsvlt
301 vlhqdwlngk eykckvsnkg lpssiektis kakgqprepq vytlppsqee mtknqvsltc
361 lvkgfypsdi avewesngqp ennykttppv ldsdgsffly srltvdksrw qegnvfscsv
421 mhealhnhyt qkslslslgk
balstilimab light chain (SEQ ID NO: 77)
    1 eivmtqspat lsvspgerat lscrasqsvs snlawyqqkp gqaprlliyg astratgipa
  61 rfsgsgsgte ftltisslqs edfavyycqq ynnwprtfgq gtkveikrtv aapsvfifpp
121 sdeqlksgta svvclInnfy preakvqwkv dnalqsgnsq esvteqdskd styslsstlt
181 lskadyekhk vyacevthqg lsspvtksfn rgec
cemiplimab heavy chain (SEQ ID NO: 78)
    1 evqllesggv lvqpggslrl scaasgftfs nfgmtwvrqa pgkglewvsg isgggrdtyf
  61 adsvkgrfti srdnskntly lqmnslkged tavyycvkwg niyfdywgqg tlvtvssast
121 kgpsvfplap csrstsesta algclvkdyf pepvtvswns galtsgvhtf pavlqssgly
181 slssvvtvps sslgtktytc nvdhkpsntk vdkrveskyg ppcppcpape flggpsvflf
241 ppkpkdtlmi srtpevtcvv vdvsqedpev qfnwyvdgve vhnaktkpre eqfnstyrvv
301 svltvlhqdw Ingkeykckv snkglpssie ktiskakgqp repqvytlpp sqeemtknqv
361 sltclvkgfy psdiavewes ngqpennykt tppvldsdgs fflysrltvd ksrwqegnvf
421 scsvmhealh nhytqkslsl slgk
cemiplimab light chain (SEQ ID NO: 79)
    1 diqmtqspss lsasvgdsit itcraslsin tflnwyqqkp gkapnlliya asslhggvps
  61 rfsgsgsgtd ftltirtlqp edfatyycqq ssntpftfgp gtvvdfrrtv aapsvfifpp
121 sdeqlksgta svvclInnfy preakvqwkv dnalqsgnsq esvteqdskd styslsstlt
181 lskadyekhk vyacevthqg lsspvtksfn rgec
dostarlimab heavy chain (SEQ ID NO: 80)
    1 evqllesggg lvqpggslrl scaasgftfs sydmswvrqa pgkglewvst isgggsytyy
  61 qdsvkgrfti srdnskntly lqmnslraed tavyycaspy yamdywgqgt tvtvssastk
121 gpsvfplapc srstsestaa lgclvkdyfp epvtvswnsg altsgvhtfp avlqssglys
181 lssvvtvpss slgtktytcn vdhkpsntkv dkrveskygp pcppcpapef lggpsvflfp
241 pkpkdtlmis rtpevtcvvv dvsqedpevq fnwyvdgvev hnaktkpree qfnstyrvvs
301 vltvlhqdwl ngkeykckvs nkglpssiek tiskakgqpr epqvytlpps qeemtknqvs
361 ltclvkgfyp sdiavewesn gqpennyktt ppvldsdgsf flysrltvdk srwqegnvfs
421 csvmhealhn hytqkslsls lgk
dostarlimab light chain (SEQ ID NO: 81)
    1 diqltqspsf lsayvgdrvt itckasqdvg tavawyqqkp gkapklliyw astlhtgvps
  61 rfsgsgsgte ftltisslqp edfatyycqh yssypwtfgq gtkleikrtv aapsvfifpp
121 sdeqlksgta svvclInnfy preakvqwkv dnalqsgnsq esvteqdskd styslsstlt
181 lskadyekhk vyacevthqg lsspvtksfn rgec
penpulimab heavy chain (SEQ ID NO: 82)
    1 evqlvesggg lvqpggslrl scaasgfafs sydmswvrqa pgkgldwvat isgggrytyy
  61 pdsvkgrfti srdnsknnly lqmnslraed talyycanry geawfaywgq gtlvtvssas
121 tkgpsvfpla psskstsggt aalgclvkdy fpepvtvswn sgaltsgvht fpavlqssgl
181 yslssvvtvp ssslgtqtyi cnvnhkpsnt kvdkkvepks cdkthtcppc papeaagaps
241 vflfppkpkd tlmisrtpev tcvvvdvshe dpevkfnwyv dgvevhnakt kpreegynst
301 yrvvsvltvl hqdwingkey kckvsnkalp apiektiska kgqprepqvy tlppsrdelt
361 knqvsltclv kgfypsdiav ewesngqpen nykttppvld sdgsfflysk ltvdksrwqq
421 gnvfscsvmh ealhnhytqk slslspgk
penpulimab light chain (SEQ ID NO: 83)
    1 diqmtqspss msasvgdrvt ftcrasqdin tylswfqqkp gkspktliyr anrlvsgvps
  61 rfsgsgsgqd ytltisslqp edmatyyclq ydefpltfga gtklelkrtv aapsvfifpp
121 sdeqlksgta svvclInnfy preakvqwkv dnalqsgnsq esvteqdskd styslsstlt
181 lskadyekhk vyacevthqg lsspvtksfn rgec
pembrolizumab heavy chain (SEQ ID NO: 84)
    1 qvqlvqsgve vkkpgasvkv sckasgytft nyymywvrqa pgqglewmgg inpsnggtnf
  61 nekfknrvtl ttdsstttay melkslqfdd tavyycarrd yrfdmgfdyw gqgttvtvss
121 astkgpsvfp lapcsrstse staalgclvk dyfpepvtvs wnsgaltsgv htfpavlqss
181 glyslssvvt vpssslgtkt ytcnvdhkps ntkvdkrves kygppcppcp apeflggpsv
241 flfppkpkdt Imisrtpevt cvvvdvsqed pevqfnwyvd gvevhnaktk preeqfnsty
301 rvvsvltvlh qdwingkeyk ckvsnkglps siektiskak gqprepqvyt lppsqeemtk
361 nqvsltclvk gfypsdiave wesngqpenn ykttppvlds dgsfflysrl tvdksrwqeg
421 nvfscsvmhe alhnhytqks lslslgk
pembrolizumab light chain (SEQ ID NO: 85)
    1 eivltqspat lslspgerat lscraskgvs tsgysylhwy qqkpgqaprl liylasyles
  61 gvparfsgsg sgtdftltis slepedfavy ycqhsrdlpl tfgggtkvei krtvaapsvf
121 ifppsdeqlk sgtasvvcll nnfypreakv qwkvdnalqs gnsqesvteq dskdstysls
181 stltlskady ekhkvyacev thqglsspvt ksfnrgec
nivolumab heavy chain (SEQ ID NO: 86)
    1 qvqlvesggg vvqpgrslrl dckasgitfs nsgmhwvrqa pgkglewvav iwydgskryy
  61 adsvkgrfti srdnskntlf lqmnslraed tavyycatnd dywgqgtlvt vssastkgps
121 vfplapcsrs tsestaalgc lvkdyfpepv tvswnsgalt sgvhtfpavl qssglyslss
181 vvtvpssslg tktytcnvdh kpsntkvdkr veskygppcp pcpapeflgg psvflfppkp
241 kdtlmisrtp evtcvvvdvs qedpevqfnw yvdgvevhna ktkpreeqfn styrvvsvlt
301 vlhqdwlngk eykckvsnkg lpssiektis kakgqprepq vytlppsqee mtknqvsltc
361 lvkgfypsdi avewesngqp ennykttppv ldsdgsffly srltvdksrw qegnvfscsv
421 mhealhnhyt qkslslslgk
nivolumab light chain (SEQ ID NO: 87)
    1 eivltqspat lslspgerat lscrasqsvs sylawyqqkp gqaprlliyd asnratgipa
  61 rfsgsgsgtd ftltisslep edfavyycqq ssnwprtfgq gtkveikrtv aapsvfifpp
121 sdeqlksgta svvclInnfy preakvqwkv dnalqsgnsq esvteqdskd styslsstlt
181 lskadyekhk vyacevthqg lsspvtksfn rgec
```

In some embodiments, the target binding domain binds PD-L1. In some embodiments, the target binding domain is derived from a commercially available anti-PDL1 antibody, antibody fragment, or derivative thereof, e.g., atezolizumab (Tecentriq®), avelumab (Bavencio®), or durvalumab (Imfinzi®), the amino acid sequences of the heavy and light chains of which are set forth in Table 3.

TABLE 3
Amino Acid Sequences of Representative anti-PD-L1 Antibody Fragments

```
atezolizumab heavy chain (SEQ ID NO: 7)
    1 evqlvesggg lvqpggslrl scaasgftfs dswihwvrqa pgkglewvaw ispyggstyy
  61 adsvkgriti sadtskntay lqmnslraed tavyycarrh wpggfdywgq gtlvtvssas
121 tkgpsvfpla psskstsggt aalgclvkdy fpepvtvswn sgaltsgvht fpavlqssgl
181 yslssvvtvp ssslgtqtyi cnvnhkpsnt kvdkkvepks cdkthtcppc papellggps
241 vflfppkpkd tlmisrtpev tcvvvdvshe dpevkfnwyv dgvevhnakt kpreeqyast
301 yrvvsvltvl hqdwlngkey kckvsnkalp apiektiska kgqprepqvy tlppsreemt
361 knqvsltclv kgfypsdiav ewesngqpen nykttppvld sdgsfflysk ltvdksrwqq
421 gnvfscsvmh ealhnhytqk slslspgk
atezolizumab light chain (SEQ ID NO: 8)
    1 diqmtqspss lsasvgdrvt itcrasqdvs tavawyqqkp gkapklliys asflysgvps
  61 rfsgsgsgtd ftltisslqp edfatyycqq ylyhpatfgq gtkveikrtv aapsvfifpp
121 sdeqlksgta svvclInnfy preakvqwkv dnalqsgnsq esvteqdskd styslsstlt
181 lskadyekhk vyacevthqg lsspvtksfn rgec
avelumab heavy chain (SEQ ID NO: 9)
    1 evqllesggg lvqpggslrl scaasgftfs syimmwvrqa pgkglewvss iypsggitfy
  61 adtvkgrfti srdnskntly lqmnslraed tavyycarik lgtvttvdyw gqgtlvtvss
121 astkgpsvfp lapsskstsg gtaalgclvk dyfpepvtvs wnsgaltsgv htfpavlqss
181 glyslssvvt vpssslgtqt yicnvnhkps ntkvdkkvep kscdkthtcp pcpapellgg
241 psvflfppkp kdtlmisrtp evtcvvvdvs hedpevkfnw yvdgvevhna ktkpreegyn
301 styrvvsvlt vlhqdwlngk eykckvsnka lpapiektis kakgqprepq vytlppsrde
361 ltknqvsltc lvkgfypsdi avewesngqp ennykttppv ldsdgsffly skltvdksrw
421 qqgnvfscsv mhealhnhyt qkslslspgk
avelumab light chain (SEQ ID NO: 10)
    1 qsaltqpasv sgspgqsiti sctgtssdvg gynyvswyqq hpgkapklmi ydvsnrpsgv
  61 snrfsgsksg ntasltisgl qaedeadyyc ssytssstrv fgtgtkvtvl gqpkanptvt
121 lfppsseelq ankatlvcli sdfypgavtv awkadgspvk agvettkpsk qsnnkyaass
181 ylsltpeqwk shrsyscqvt hegstvektv aptecs
durvalumab heavy chain (SEQ ID NO: 11)
    1 evqlvesggg lvqpggslrl scaasgftfs rywmswvrqa pgkglewvan ikqdgsekyy
  61 vdsvkgrfti srdnaknsly lqmnslraed tavyycareg gwfgelafdy wgqgtlvtvs
121 sastkgpsvf plapssksts ggtaalgclv kdyfpepvtv swnsgaltsg vhtfpavlqs
181 sglyslssvv tvpssslgtq tyicnvnhkp sntkvdkrve pkscdkthtc ppcpapefeg
241 gpsvflfppk pkdtlmisrt pevtcvvvdv shedpevkfn wyvdgvevhn aktkpreegy
301 nstyrvvsvl tvlhqdwlng keykckvsnk alpasiekti skakgqprep qvytlppsre
361 emtknqvslt clvkgfypsd iavewesngq pennykttpp vldsdgsffl yskltvdksr
421 wqqgnvfscs vmhealhnhy tqkslslspg k
durvalumab heavy chain (SEQ ID NO: 12)
    1 eivltqspgt lslspgerat lscrasqrvs ssylawyqqk pgqaprlliy dassratgip
  61 drfsgsgsgt dftltisrle pedfavyycq qygslpwtfg qgtkveikrt vaapsvfifp
121 psdeqlksgt asvvclInnf ypreakvqwk vdnalqsgns qesvteqdsk dstyslsstl
181 tlskadyekh kvyacevthq glsspvtksf nrgec
```

In some embodiments, the target binding domain binds the cognate ligand the cognate receptor of an antigen. Where the cognate receptor or cognate ligand is naturally present on an immune cell. Therefore, by binding the cognate receptor or cognate ligand with the target binding domain positions the mabkine in close proximity to the cell surface of the immune cell, enabling binding of the mabkine IL-15 domain to an IL-15R on the immune cell.

In some embodiments, the target binding domain binds a cognate ligand or cognate receptor of an antigen. In some embodiments, target binding domain is a scFv that binds NKG2A. In some embodiments, the target binding domain is derived from the sequence of a commercially available anti-NKG2A antibody, antibody fragment, or variant thereof, for example, monalizumab (formerly IPH2201) and humanized Z199; the amino acid sequences of the heavy and light chains of which are set forth in Table 4.

TABLE 4
Amino acid Sequences of Representative anti-NKG2A Antibody Fragments

```
monalizumab heavy chain (SEQ ID NO: 13)
    1 evqlvqsgae vkkpgeslki sckgsgysft sywmnwvrqm pgkglewmgr idpydsethy
  61 spsfqgqvti sadksistay lqwsslkasd tamyycargg ydfdvgtlyw ffdvwgqgtt
121 vtvssastkg psvfplapcs rstsestaal gclvkdyfpe pvtvswnsga ltsgvhtfpa
181 vlqssglysl ssvvtvpsss lgtktytcnv dhkpsntkvd krveskygpp cppcpapefl
241 ggpsvflfpp kpkdtlmisr tpevtcvvvd vsqedpevqf nwyvdgvevh naktkpreeq
301 fnstyrvvsv ltvlhqdwln gkeykckvsn kglpssiekt iskakgqpre pqvytippsq
361 eemtknqvsl tclvkgfyps diavewesng qpennykttp pvldsdgsff lysrltvdks
421 rwqegnvfsc svmhealhnh ytqkslslsl gk
monalizumab light chain (SEQ ID NO: 14)
    1 diqmtqspss lsasvgdrvt itcraseniy sylawyqqkp gkapklliyn aktlaegvps
  61 rfsgsgsgtd ftltisslqp edfatyycqh hygtprtfgg gtkveikrtv aapsvfifpp
121 sdeqlksgta svvclInnfy preakvqwkv dnalqsgnsq esvteqdskd styslsstlt
181 lskadyekhk vyacevthqg lsspvtksfn rgec
humanized Z199 heavy chain (SEQ ID NO: 15)
    1 evqlvesggg lvkpggslrl scaasgftfs syamswvrqa pgkglewvse issggsytyy
  61 adsvkgrfti srdnaknsly lqmnslraed tavyycarhg dyprffdvwg qgttvtvss
humanized Z199 light chain (SEQ ID NO: 16)
    1 eivltqspat lslspgerat lscsasssvs syiywyqqkp gqaprlliyl tsnlasgipa
  61 rfsgsgsgtd ftltisslep edfavyycqq wsgnpytfgq gtkleik
```

In some embodiments, the target binding domain contains a VL having the amino acid sequence set forth below (SEQ ID NO: 17):

```
  1 diqmtqspas lsasvgetvt itcraseniy
      sylawyqqkq gkspqflvyn aktlaegvps
61 rfsgsgsgtq fslkinslqp edfgsyycqh
      hygtprtfgg qtkleik
```

Additionally, the target binding domain contains a VH having the amino acid sequence set forth below (SEQ ID NO: 18):

```
    1 qvqlqqpgae lvrpgasvkl sckasgytft
        sywmnwvkqr peqglqwigr idpydsethy
  61 sqkfkdkail tvdkssstay mrlssltsed
        savyycargg ydfdvgtlyw ffdvwgagtt
121 vtvss
```

### Hinge Domain

The hinge domain connects the target binding domain to the IL-15Rα domain. A hinge domain may provide flexibility in terms of allowing the target binding domain to obtain an optimal orientation for binding, enhancing immune cell stimulation activities.

In some embodiments, the hinge domain is derived from IgA, IgD, IgE, IgG, or IgM. In some embodiments, the hinge domain is derived from CD3ζ, CD4, CD8α, CD28, IgG1, IgG2, or IgG4. Amino acid sequences of representative hinge domains of which are listed in Table 5

TABLE 5
Amino Acid Sequences of Representative
Hinge Domains

| Hinge domain | Sequence |
|---|---|
| CD3ζ (SEQ ID NO: | QSFGLLDPK |
| 19) | |
| CD4 (SEQ ID NO: 20) | LSEGDKVKMDSRIQVLSRGVNQT |
| CD8α (SEQ ID NO: | KPTTTPAPRPPTPAPTIASQPLS |
| 21) | KRPEACRPAAGGAVHTRGLDFAC |
| | DIY |
| CD28 (SEQ ID NO: | IEVMYPPPYLDNERSNGTIIHVK |
| 22) | GKHLCPSPLFPGPSKP |
| IgG1 (SEQ ID NO: 23) | DKTHTCPPCPAPELLGGP |
| IgG2 (SEQ ID NO: 24) | ERKCCVECPPCPAPPAAA |
| IgG4 (SEQ ID NO: 25) | PPCPPCPAPEFLGGP |
| IgG4 S5P (SEQ ID | PPCPSCPAPEFLGGP |
| NO: 26) | |

A hinge domain derived from IgG4 enables two mabkine fusion proteins to dimerize, forming a homodimer. Antibodies from the IgG4 subclass have the ability to perform Fab-arm exchange, where the disulfide bonds are reduced under redox conditions, and reform between two new molecules, potentially creating bi-specific antibodies. In some embodiments, the IgG4-derived hinge domain is modified to prevent Fab-arm exchange. In some embodiments, the IgG4-derived hinge domain has a serine to proline substitution at position 5 (S5P) resulting in SEQ ID NO: 26 (which corresponds to position 228 (S228P) when numbered according to Kabat numbering).

### IL-15Rα Domain

IL-15 stimulates CD8+ T cell and natural killer (NK) cell activation, proliferation, and cytolytic activity. The mabkine contains an IL-15Rα domain, a linker, and an IL-15 domain that, together, bind to IL-15R on immune cells (e.g., CD8+ T cells and NK cells).

The IL-15Rα domain may be derived from the human Interleukin 15 Receptor Subunit Alpha (IL-15RA) gene. IL-15RA encodes a cytokine receptor that specifically binds IL-15 with high affinity. IL15Rα is capable of binding IL15 independent of other subunits (e.g., IL-15Rß or IL-15Rγ). The nucleic acid sequence of the IL15RA gene is provided at NCBI Accession No. NM_001256765, version NM_001256765.1. The amino acid sequence of a representative IL-15Rα is provided at NCBI Accession No. NP_001243694, version NP_001243694.1, incorporated herein by reference and set forth below (SEQ ID NO: 27):

```
    1 mrlagrqvpe qrsppppglg sarpgspavs
        cgaaamaprr argcrtlglp alllllllrp
  61 patrdardrl avlagrsris esinhevqth
        eacvrlrtme ncpqchhhrt srqqagitcp
121 ppmsvehadi wvksyslysr eryicnsgfk
        rkagtsslte cvlnkatnva hwttpslkci
181 rdpalvhqrp appstvttag vtpqpeslsp
        sgkepaassp ssnntaatta aivpgsqlmp
241 skspstgtte isshesshgt psqttaknwe
        ltasashqpp gvypqghsdt tvaiststvl
301 lcglsavsll acylksrqtp plasvemeam
        ealpvtwgts srdedlencs hhl
```

The Sushi domain of IL-15Rα is the minimal region required for IL-15 binding activity. Sushi domains, which are common motifs in protein-protein interactions, contain four cysteines forming two disulfide bonds in a 1-3 and 2-4 pattern. Amino acid substitution of the first or fourth cysteine in IL-15Rα substantially abolishes its IL-15 binding activity. In some embodiments, the IL-15Rα domain contains the IL-15Rα Sushi domain having the amino acid sequence represented in bold in SEQ ID NO: 27, which is set forth below (SEQ ID NO: 28).

```
  1 itcpppmsve hadiwvksys lysreryicn
      sgfkrkagts sltecvlnka tnvahwttps
61 lkcir
```

### IL-15 Domain

The IL-15 domain may also be derived from the human IL15 gene. The human IL-15 locus consists of 9 exons and 8 introns. Two IL-15 isoforms have been described, the classical long signal peptide (LSP) isoform and an alternative short signal peptide (SSP) isoform; both isoforms encode the identical IL-15 mature protein that has 114 amino acids. The LSP isoform has a 48 long signal peptide, while the SSP isoform has a 21 long signal peptide. These signal (or leader) peptides determine the intracellular localization and potential secretion of the associated mature protein. The 114 amino acid long mature IL-15 protein is encoded by exons 5 to 8 of the IL15 gene.

The nucleic acid sequence of the IL15 gene is provided at NCBI Accession No. NM_000585, version NM_000585.5. The amino acid sequence of a representative IL-15 LSP isoform is provided at NCBI Accession No. NP_000576, version NP_000576.1, incorporated herein by reference and set forth below (SEQ ID NO: 29):

```
    1 mriskphlrs isiqcylcll Inshflteag
        ihvfilgcfs aglpkteanw vnvisdlkki
  61 edliqsmhid atlytesdvh psckvtamkc
        fllelqvisl esgdasihdt venliilann
121 slssngnvte sgckeceele eknikeflqs
        fvhivqmfin ts
```

The amino acid sequence of a representative IL-15 SSP isoform is provided at NCBI Accession No. NP_751915, version NP_751915.1, incorporated herein by reference and set forth below (SEQ ID NO: 30):

```
    1 mvlgtidlcs cfsaglpkte anwvnvisdl
        kkiedliqsm hidatlytes dvhpsckvta
  61 mkcfllelqv islesgdasi hdtvenliil
        annslssngn vtesgckece eleeknikef
121 lqsfvhivqm fints
```

The amino acid sequence of a representative mature IL-15 protein is set forth below (SEQ ID NO: 31):

| | |
|---|---|
| 1 | nwvnvisdlk kiedliqsmh idatlytesd vhpsckvtam kcfllelqvi slesgdasih |
| 61 | 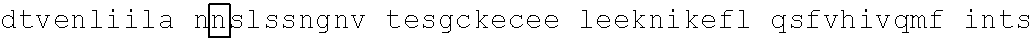 |

In some embodiments, the IL-15 domain is the mature IL-15 protein. In some embodiments, the IL-15 domain is the one of the LSP or SSP full-length IL-15 (e.g., SEQ ID NOs: 29 or 30). In some embodiments, the IL-15 domain has an amino acid substitution at position 72, when numbered according to SEQ ID NO: 31. In some embodiments, the substitution is an asparagine to aspartic acid substitution at position 72 (N72D).

The mabkine further contains a linker disposed between the IL-15Rα domain and the IL-15 domain. A linker may provide flexibility in terms of allowing the IL-15Rα domain to bind to the IL-15 domain. A linker contains at least four amino acids, the selection of which is not critical.

In some embodiments, the linker contains an amino acid having the sequence GGGX, GGGGX (SEQ ID NO: 32), or GSSGSX (SEQ ID NO: 33), where X is any nucleotide, typically either cysteine (C) or serine(S), or repeating sequence thereof. In some embodiments, the linker has the amino acid sequence GGGGS (SEQ ID NO: 34), GGGGGGGGS (SEQ ID NO: 35), GGGGSGGGGSGGGGS (SEQ ID NO: 36), GGGGSGGGGSGGGGSGGGGS (SEQ ID NO: 37), SGGSGGGGSGGGSGGGGSLQ (SEQ ID NO: 38), GSTSGSGKPGSGEGSTKG (SEQ ID NO: 39), KESGSVSSEQLAQFRSLD (SEQ ID NO: 40), EGKSSGSGSESKST (SEQ ID NO: 41), or GSAGSAAGSGEF (SEQ ID NO: 42).

In some embodiments, the mabkine has a Fc (fragment crystallizable) domain derived from an antibody. A Fc domain provides additional stability to the mabkine. The Fc domain may also be used in purification, isolation, or detection methods. In some embodiments, the Fc region is derived from a constant heavy (CH) 3 domain. In some embodiments, the Fc region is derived from the IgG4 antibody subclass. IgG4 subclass Fc domains do not induce antibody-dependent cellular cytotoxicity (ADCC) or complement-dependent cytotoxicity (CDC). In some embodiments, the Fc region has the amino acid sequence set forth below as SEQ ID NO: 43:

```
    1 svflfppkpk dtlmisrtpe vtcvvvdvsq
        edpevqfnwy vdgvevhnak tkpreeqfns
  61 tyrvvsvltv lhqdwlngke ykckvsnkgl
        pssiektisk akgqprepqv ytlppsqeem
121 tknqvsltcl vkgfypsdia vewesngqpe
        nnykttppvl dsdgsfflys rltvdksrwq
181 egnvfscsvm healhnhytq kslslslgk
```

In some embodiments, the mabkine includes a signal peptide disposed N-terminal to the target binding domain. The term “signal peptide” as used herein refers to a short (e.g., 5-100 such as a 10-100 or 5-30 amino acid-long) stretch of amino acids that directs the transport of the mabkine within a cell. Mabkines containing a signal peptide domain will be secreted from the cell.

In some embodiments, the signal peptide is derived from albumin, CD8α, CD33, erythropoietin (EPO), IL-2, human or mouse Ig-kappa chain V-III (IgK VIII), tissue plasminogen activator (tPA), or secreted alkaline phosphatase (SEAP). Signal peptides may also be synthetic (i.e., non-naturally occurring). Amino acid sequences of representative signal peptides are listed in Table 6.

TABLE 6
Amino Acid Sequences of
Representative Signal Peptides

| Signal peptide | Sequence |
|---|---|
| Albumin | MKWVTFISLLFLFSSAYS |
| (SEQ ID NO: 44) | |
| Synthetic, modified | MKWVTFISLLFLFSSSSRA |
| albumin | |
| (SEQ ID NO: 45) | |
| CD8α (SEQ ID NO: 46) | MALPVTALLLPLALLLHAARP |
| CD33 (SEQ ID NO: 47) | MPLLLLLPLLWAGALA |
| EPO (SEQ ID NO: 48) | MGVHECPAWLWLLLSLLSLPL |
| | GLPVLG |
| IL-2 (SEQ ID NO: 49) | MYRMQLLSCIALSLALVTNS |
| Mouse IgK VIII | METDTLLLWVLLLWVPGSTG |
| (SEQ ID NO: 50) | |
| Human IgK VIII | MEAPAQLLFLLLLWLPDTTG |
| (SEQ ID NO: 51) | |
| Synthetic, | MEAPAQLLFLLLLWLPSSRA |
| modified human | |
| IgK VIII | |
| (SEQ ID NO: 52) | |
| tPA (SEQ ID NO: 53) | MDAMKRGLCCVLLLCGAVFVSPS |
| SEAP (SEQ ID NO: 54) | MLLLLLLLGLRLQLSLG |
| Synthetic consensus | MLLLLLLLLLLALALA |
| (SEQ ID NO: 55) | |
| Synthetic secrecon | MWWRLWWLLLLLLLLWPMVWA |
| (SEQ ID NO: 56) | |

In some embodiments, the mabkine contains a selection marker to aid in isolation, capture or detection. A selection marker typically entails addition of in-frame nucleic acids that will be translated into amino acids along with the protein to which the tag is attached. Representative examples of purification tags include polyhistidine, FLAG epitope, histidine affinity tag (HAT), herpes simplex virus (HSV) epitope, human influenza hemagglutinin (HA), glutathione S-transferase (GST), KT3 epitope, maltose binding protein (MBP), AU1 epitope, AU5 epitope, Bacteriophage T7 epitope, myc tags. Amino acid sequences of representative purification tags are listed in Table 7.

TABLE 7
Amino Acid Sequences of Representative
Selection Markers

| Selection Marker | Sequence |
|---|---|
| AUl cpitopc (SEQ ID NO: 57) | DTYRYI |
| AU5 epitope (SEQ ID NO: 58) | TDFYLK |
| Polyhistidine (SEQ ID NO: 59) | HHHHHH |
| FLAG epitope (SEQ ID NO: 60) | DYKDDDK |
| FLAG His tag (SEQ ID NO: 61) | EFDYKDDDDKGS |
| | LPETGGHHHHHH |
| HAT (SEQ ID NO: 62) | KDHLIHNVHKEF |
| | HAHAHNK |
| HSV epitope (SEQ ID NO: 63) | QPELAPED |

In some embodiments, the selection marker is a Polyhistidine selection marker (SEQ ID NO: 59), commonly referred to as a His tag. In some embodiments, the selection marker is N-terminal to the target binding domain.

In some embodiments, the mabkine (also referred to herein as Mabkine #1) has the amino acid sequence set forth below (SEQ ID NO: 64), and that contains the following elements, from N-terminus to C-terminus, an IL-2 signal peptide, an anti-NKG2A scFv, an IgG4 hinge, an IgG4 Fc, an IL-15Rα Sushi domain, a linker, a mature IL-15 (which lacks a IL-15 signal peptide) set forth in Table 8:

| | |
|---|---|
| 1 | myrmqllsci alslalvins digmtqspas |
| | lsasvgetvt itcraseniy sylawyqqkg |
| 61 | gkspqflvyn aktlaegvps rfsgsgsgtq |
| | fslkinslgp edfgsyycqh hygtprtfgg |
| 121 | gtkleikggg gsggggsggg gsqvqlqqpg |
| | aelvrpgasv klsckasgyt ftsywmnwvk |
| 181 | grpeqglqwi gridpydset hysqkfkdka |
| | iltvdkssst aymilsslts edsavyycar |
| 241 | ggydfdvgtl ywifdvwgag ttvtvssppc |
| | ppcpapeflg gpsvflfppk pkdtlmisrt |
| 301 | pevtcvvvdv sqedpevqfn wyvdgvevhn |
| | aktkpreeqf nstyrvysvl tvlhqdwing |
| 361 | keykckvsnk gipssiekti skakgqprep |
| | qvytippsqe emtknqvslt clvkgfypsd |
| 421 | iavewesngq pennykttpp vldsdgsffl |
| | ysrltvdksr wqegnviscs vmhealhnhy |
| 481 | tqkslsislg kitcpppmsv ehadiwvksy |
| | slysreryic nsgfkrkagt ssltecvink |
| 541 | atnvahwttp sikcirsggs ggggggggsg |
| | gggslqnwvn visdlkkied liqsmhidat |
| 601 | lytesdvhps ckytamkcfl lelqvisles |
| | gdasihdtve nliilannsl ssngnvtesg |
| 661 | ckeceeleek nikeflqsfv hivqmfints |

TABLE 8
Amino Acid Sequences of the Elements of Mabkine #1

```
IL-2 signal peptide (SEQ ID NO: 49)
    1 myrmqllsci alslalvtns
anti-NKG2A VL (SEQ ID NO: 17)
    1 diqmtqspas lsasvgetvt itcraseniy sylawyqqkg gkspqflvyn aktlaegvps
  61 rfsgsgsgtq fslkinslqp edfgsyycqh hygtprtfgg gtkleik
Linker (SEQ ID NO: 36)
    1 ggggsggggs ggggs
anti-NKG2A VH (SEQ ID NO: 18)
    1 qvqlqqpgae lvrpgasvkl sckasgytft sywmnwvkqr peqglqwigr idpydsethy
  61 sqkfkdkail tvdkssstay mrlssltsed savyycargg ydfdvgtlyw ffdvwgagtt
121 vtvss
IgG4 hinge (SEQ ID NO: 25)
    1 ppcppcpape flggp
IgG4 Fc (SEQ ID NO: 43)
    1 svflfppkpk dtlmisrtpe vtcvvvdvsq edpevqfnwy vdgvevhnak tkpreeqfns
  61 tyrvvsvltv lhqdwingke ykckvsnkgl pssiektisk akgqprepqv ytlppsqeem
121 tknqvsltcl vkgfypsdia vewesngqpe nnykttppvl dsdgsfflys rltvdksrwq
181 egnvfscsvm healhnhytq kslslslgk
IL-15Rα Sushi domain (SEQ ID NO: 28)
    1 itcpppmsve hadiwvksys lysreryicn sgfkrkagts sltecvlnka tnvahwttps
  61 1kcir
linker (SEQ ID NO: 38)
    1 sggsggggsg ggsggggslq
mature IL-15 (without signal peptide) (SEQ ID NO: 31)
    1 nwvnvisdlk kiedliqsmh idatlytesd vhpsckvtam kcfllelqvi slesgdasih
  61 dtvenliila nnslssngnv tesgckecee leeknikefl qsfvhivqmf ints
```

In some embodiments, the mabkine (also referred to herein as Mabkine #2) has the amino acid sequence set forth below (SEQ ID NO: 65), and that contains the following elements, from N-terminus to C-terminus, an IL-2 signal peptide, a His tag selection marker, an anti-NKG2A scFv, a IgG4 hinge, a IgG4 Fc, a IL-15Rα Sushi domain, a linker, a mature IL-15 (which lacks a IL-15 signal peptide) set forth below in Table 9:

| | |
|---|---|
| 1 | myrmqllsci alslalvtns hhhhhhvvhd iqmtqspasl sasvgetvti tcraseniys |
| 61 | ylawyqqkqg kspqflvyna ktlaegvpsr fsgsgsgtqf slkinslqpe dfgsyycqhh |
| 121 | ygtprtfggg tkleikgggg sggggsgggg sqvqlqqpga elvrpgasvk lsckasgytf |
| 181 | tsywmnwvkq rpeqglqwig ridpydseth ysqkfkdkai ltvdksssta ymrlssltse |
| 241 | dsavyycarg gydfdvgtly wffdvwgagt tvtvssppcp pcpapeflgg psvflfppkp |
| 301 | kdtlmisrtp evtcvvvdvs qedpevqfnw yvdgvevhna ktkpreeqfn styrvvsvlt |
| 361 | vlhqdwlngk eykckvsnkg lpssiektis kakgqprepq vytlppsqee mtknqvsltc |
| 421 | lvkgfypsdi avewesngqp ennykttppv ldsdgsffly srltvdksrw qegnvfscsv |
| 481 | mhealhnhyt qkslslslgk itcpppmsve hadiwvksys lysreryicn sgfkrkagts |
| 541 | sltecvlnka tnvahwttps lkcirsggsg ggggggsgg ggslqnwvnv isdlkkiedl |
| 601 | iqsmhidatl ytesdvhpsc kvtamkcfll elqvislesg dasihdtven liilannsls |
| 661 | sngnvtesgc keceeleekn ikeflqsfvh ivqmfints |

TABLE 9
Amino Acid Sequences of the Elements of Mabkine #2

```
IL-2 signal peptide (SEQ ID NO: 49)
    1 myrmqllsci alslalvtns
His tag selection marker (SEQ ID NO: 59)
    1 hhhhhh
anti-NKG2A VL (SEQ ID NO: 17)
    1 digmtqspas lsasvgetvt itcraseniy sylawyqqkg gkspqflvyn aktlaegvps
  61 xfsgsgsgtq fslkinslqp edfgsyycqh hygtprtfgg gtkleik
Linker (SEQ ID NO: 36)
    1 ggggsggggs ggggs
anti-NKG2A VH (SEQ ID NO: 18)
    1 qvqlqqpgae lvrpgasvkl sckasgytft sywmnwvkqr peqglqwigr idpydsethy
  61 sqkfkdkail tvdkssstay mrlssltsed savyycargg ydfdvgtlyw ffdvwgagtt
121 vtvss
IgG4 hinge (SEQ ID NO: 25)
    1 ppcppcpape flggp
IgG4 Fc (SEQ ID NO: 43)
    1 svflfppkpk dtlmisrtpe vtcvvvdvsq edpevqfnwy vdgvevhnak tkpreeqfns
  61 tyrvvsvltv lhqdwlngke ykckvsnkgl pssiektisk akgqprepqv ytlppsqeem
121 tknqvsltcl vkgfypsdia vewesngqpe nnykttppvl dsdgsfflys rltvdksrwq
181 egnvfscsvm healhnhytq kslslslgk
IL-15Rα Sushi domain (SEQ ID NO: 28)
    1 itcpppmsve hadiwvksys lysreryicn sgfkrkagts sltecvlnka tnvahwttps
  61 1kcir
GS linker (SEQ ID NO: 38)
    1 sggsggggsg ggsggggslq
mature IL-15 (without signal peptide) (SEQ ID NO: 31)
    1 nwvnvisdlk kiedliqsmh idatlytesd vhpsckvtam kcfllelqvi slesgdasih
  61 dtvenliila nnslssngnv tesgckecee leeknikefl qsfvhivqmf ints
```

In some embodiments, the mabkine (also referred to herein as Mabkine #3) has the amino acid sequence set forth below (SEQ ID NO: 66), and that contains the elements, from N-terminus to C-terminus, an IL-2 signal peptide, an anti-NKG2A scFv, an IgG1 hinge, an IL-15Rα Sushi domain, a linker, a mature IL-15 (which lacks a IL-15 signal peptide) set forth in Table 10:

```
    1 myrmqllsci alslalvtns hhhhhhvvhd
        iqmtqspasl sasvgetvti tcraseniys
  61 ylawyqqkqg kspqflvyna ktlaegvpsr
        fsgsgsgtqf slkinslqpe dfgsyycqhh
121 ygtprtfggg tkleikgggg sggggsgggg
        sqvqlqqpga elvrpgasvk lsckasgytf
181 tsywmnwvkq rpeqglqwig ridpydseth
        ysqkfkdkai ltvdksssta ymrlssltse
241 dsavyycarg gydfdvgtly wffdvwgagt
        tvtvssdkth toppcpapel lggpitoppp
301 msvehadiwv ksyslysrer yicnsgfkrk
        agtssltecv Inkatnvahw ttpslkcirs
361 ggsggggsgg gsggggslan wvnvisdlkk
        iedliqsmhi datlytesdv hpsckvtamk
421 cfllelqvis lesgdasihd tvenliilan
        nslssngnvt esgckeceel eeknikeflq
481 sfvhivqmfi nts
```

TABLE 10
Ammino Acid Sequences of the Elements of Mabkine #3

IL-2 signal peptide (SEQ ID NO: 49)

| | |
|---|---|
| 1 | myrmqllsci alslalvtns |

His tag selection marker (SEQ ID NO: 59)

| | |
|---|---|
| 1 | hhhhhh |

anti-NKG2A VL (SEQ ID NO: 17)

| | |
|---|---|
| 1 | diqmtqspas lsasvgetvt itcraseniy sylawyqqkq gkspqflvyn aktlaegvps |
| 61 | rfsgsgsgtq fslkinslop edfgsyycqh hygtprtfgg gtkleik |

Linker (SEQ ID NO: 36)

| | |
|---|---|
| 1 | ggggsggggs ggggs |

anti-NKG2A VH (SEQ ID NO: 18)

| | |
|---|---|
| 1 | qvqlqqpgae lvrpgasvkl sckasgytft sywmnwvkqr peqglqwigr idpydsethy |
| 61 | sqkfkdkail tvdkssstay mrlssltsed savyycargg ydfdvgtlyw ffdvwgagtt |
| 121 | vtvss |

IgG1 hinge (SEQ ID NO: 23)

| | |
|---|---|
| 1 | dkthtcppcp apellggp |

IL-15Ra Sushi domain (SEQ ID NO: 28)

| | |
|---|---|
| 1 | itcpppmsve hadiwvksys lysreryicn sgfkrkagts sltecvlnka tnvahwttps |
| 61 | lkcir |

GS linker (SEQ ID NO: 38)

| | |
|---|---|
| 1 | sggsggggsg ggsggggslq |

mature IL-15 (without signal peptide) (SEQ ID NO: 31)

| | |
|---|---|
| 1 | nwvnvisdlk kiedliqsmh idatlytesd vhpsckvtam kcfllelqvi slesgdasih |
| 61 | 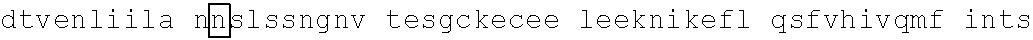 |

### Nucleic Acids

In one aspect, the disclosure provides a nucleic acid that encodes an mabkine. The term “nucleic acid” as used herein refers to a polymer of nucleotides, each of which are organic molecules consisting of a nucleoside (a nucleobase and a five-carbon sugar) and a phosphate. The term nucleotide, unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide.

In some embodiments, the sequence of the nucleic acid encoding the mabkine may be codon-optimized to match balance frequency of codon usage in the organism and cell type (e.g., a human immune cell) expressing the mabkine. The use of efficient codons increases elongation rate. In some embodiments, the nucleic acid sequence is designed to exclude stretches of 20 nucleic acids of direct or inverted repeat sequences. Direct repeats are nucleotides sequences that consists of two or more repeats of a specific sequence, such that the repeats are present in multiple copies of a larger sequence. Generally, a direct repeat occurs when a sequence is repeated with the same pattern downstream. An inverted repeat is a single stranded sequence of nucleotides followed downstream by its reverse complement.

In some embodiments, the sequence of the nucleic acid encoding the mabkine is designed to exclude putative alternate splice sites. See, Wang et al., Gene 366:219-27 (2006), Lee et al., Annu. Rev. Biochem. 84:291-323 (2015), Baharlou et al., Sci. Rep. 8:5063-11 (2018), Jaganathan et al., Cell 176:535-548 (2019). In some embodiments, 3′ ends are modified to ensure the presence of proper transcription termination consensus sequences.

In some embodiments, the nucleic acids encoding an mabkine contain a 5′ untranslated region (UTR), which may provide optimal cloning and expression in a cell line (e.g., CHO-S cells). The nucleic acid sequence of a representative 5′ UTR is set forth below (SEQ ID NO: 67):

```
  1 gctagcgcta ccggactcag atctcgagct
      caagcttcga attctgcagt cgacggtacc
61 gcgggcccgg gatccgccac c
```

In some embodiments, the mabkine-encoding nucleic acid has the nucleic acid sequence set forth below (SEQ ID NO: 68), which encodes Mabkine #1 (SEQ ID NO: 64) that contains the elements set forth in Table 8.

| | |
|---|---|
| 1 | atgtacagga tgcaactcct gtcttgcatt gcactaagtc ttgcacttgt cacgaattcg |
| 61 | gacatccaga tgactcagtc tccagcctcc ctatctgcat ctgtgggaga aactgtcacc |
| 121 | atcacgtgtc gagcaagtga gaatatttac agttatttag catggtatca gcagaaacag |
| 181 | ggaaaatctc ctcagttctt ggtctataat gcaaaaacct tagcagaagg tgtgccatca |
| 241 | aggttcagtg gcagtggatc aggcacacag ttttctctga agatcaacag cctacagcct |
| 301 | gaagattttg ggagttatta ctgtcaacat cactatggta ctcctcggac gttcggtgga |
| 361 | ggcaccaagc tggaaatcaa aggtggaggt ggcagcggag gaggtgggtc cggcggtgga |
| 421 | ggaagccagg tccaactgca gcagcctggg gctgagctgg tgaggcctgg ggcttcagtg |
| 481 | aagctgtcct gcaaggcttc tggctacacg ttcaccagct actggatgaa ctgggttaag |
| 541 | cagaggcctg agcaaggcct tcagtggatt ggaaggattg atccttacga tagtgaaact |
| 601 | cactacagtc aaaagttcaa ggacaaggcc atattgactg tagacaaatc ctccagcaca |
| 661 | gcctacatgc gactcagcag cctgacatct gaggactctg cggtctatta ctgtgcaaga |
| 721 | gggggctatg atttcgacgt aggaactctc tactggttct tcgatgtctg gggcgcaggg |
| 781 | accacggtca ccgtctcctc acccccatgc ccaccgtgcc cagcacctga gttcctgggg |
| 841 | ggaccatcag tcttcctgtt ccccccaaaa cccaaggaca ctctcatgat ctcccggacc |
| 901 | cctgaggtca cgtgcgtggt ggtggacgtg agccaggaag accccgaggt ccagttcaac |
| 961 | tggtacgtgg atggcgtgga ggtgcataat gccaagacaa agccgcggga ggagcagttc |
| 1021 | aacagcacgt accgtgtggt cagcgtcctc accgtcctgc accaggactg gctgaacggc |
| 1081 | aaggagtaca agtgcaaggt ctccaacaaa ggcctcccgt cctccatcga gaaaaccatc |
| 1141 | tccaaagcca aagggcagcc ccgagagcca caggtgtaca ccctgccccc atcccaggag |
| 1201 | gagatgacca agaaccaggt cagcctgacc tgcctggtca aaggcttcta ccccagcgac |
| 1261 | atcgccgtgg agtgggagag caatgggcag ccggagaaca actacaagac cacgcctccc |
| 1321 | gtgctggact ccgacggctc cttcttcctc tacagcaggc taaccgtgga caagagcagg |
| 1381 | tggcaggagg ggaatgtctt ctcatgctcc gtgatgcatg aggctctgca caaccactac |
| 1441 | acacagaaga gcctctccct gtctctgggt aaaatcacgt gccctccccc catgtccgtg |
| 1501 | gaacacgcag acatctgggt caagagctac agcttgtact ccagggagcg gtacatttgt |
| 1561 | aactctggtt tcaagcgtaa agccggcacg tccagcctga cggagtgcgt gttgaacaag |
| 1621 | gccacgaatg tcgcccactg gacaaccccc agtctcaaat gcattagaag cggcggcagc |
| 1681 | ggcggcggcg gcagcggcgg cggcagcggc ggcggcggca gcctgcagaa ctgggtgaat |
| 1741 | gtaataagtg atttgaaaaa aattgaagat cttattcaat ctatgcatat tgatgctact |
| 1801 | ttatatacgg aaagtgatgt tcaccccagt tgcaaagtaa cagcaatgaa gtgctttctc |
| 1861 | ttggagttac aagttatttc acttgagtcc ggagatgcaa gtattcatga tacagtagaa |
| 1921 | aatctgatca tcctagcaaa caacagtttg tottctaatg ggaatgtaac agaatctgga |
| 1981 | tgcaaagaat gtgaggaact ggaggaaaaa aatattaaag aatttttgca gagttttgta |
| 2041 | catattgtcc aaatgttcat caacacttct taa |

In some embodiments, the mabkine-encoding nucleic acid has the nucleic acid sequence set forth below (SEQ ID NO: 69), which encodings Mabkine #2 (SEQ ID NO: 65) that contains the elements set forth in Table 9.

| | |
|---|---|
| 1 | atgtacagga tgcaactcct gtcttgcatt gcactaagtc ttgcacttgt cacgaattcg |
| 61 | catcaccatc accatcacga catccagatg actcagtctc cagcctccct atctgcatct |
| 121 | gtgggagaaa ctgtcaccat cacatgtcga gcaagtgaga atatttacag ttatttagca |
| 181 | tggtatcagc agaaacaggg aaaatctcct cagttcttgg tctataatgc aaaaacctta |
| 241 | gcagaaggtg tgccatcaag gttcagtggc agtggatcag gcacacagtt ttctctgaag |
| 301 | atcaacagcc tacagcctga agattttggg agttattact gtcaacatca ctatggtact |
| 361 | cctcggacgt tcggtggagg caccaagctg gaaatcaaag gtggaggtgg cagcggagga |
| 421 | ggtgggtccg gcggtggagg aagccaggtc caactgcagc agcctggggc tgagctggtg |
| 481 | aggcctgggg cttcagtgaa gctgtcctgc aaggcttctg gctacacgtt caccagctac |
| 541 | tggatgaact gggttaagca gaggcctgag caaggccttc agtggattgg aaggattgat |
| 601 | ccttacgata gtgaaactca ctacagtcaa aagttcaagg acaaggccat attgactgta |
| 661 | gacaaatcct ccagcacagc ctacatgcga ctcagcagcc tgacatctga ggactctgcg |
| 721 | qtctattact gtgcaagagg gggctatgat ttcgacgtag gaactctcta ctggttcttc |
| 781 | gatgtctggg gcgcagggac cacggtcacc gtctcctcac ccccatgccc accgtgccca |
| 841 | qcacctgagt tcctgggggg accatcagtc ttcctgttcc ccccaaaacc caaggacact |
| 901 | ctcatgatct cccggacccc tgaggtcacg tgcgtggtgg tggacgtgag ccaggaagac |
| 961 | cccgaggtcc agttcaactg gtacgtggat ggcgtggagg tgcataatgc caagacaaag |
| 1021 | ccgcgggagg agcagttcaa cagcacgtac cgtgtggtca gcgtcctcac cgtcctgcac |
| 1081 | caggactggc tgaacggcaa ggagtacaag tgcaaggtct ccaacaaagg cctcccgtcc |
| 1141 | tccatcgaga aaaccatctc caaagccaaa gggcagcccc gagagccaca ggtgtacacc |
| 1201 | ctgcccccat cccaggagga gatgaccaag aaccaggtca gcctgacctg cctggtcaaa |
| 1261 | ggcttctacc ccagcgacat cgccgtggag tgggagagca atgggcagcc ggagaacaac |
| 1321 | tacaagacca cgcctcccgt gctggactcc gacggctcct tcttcctcta cagcaggcta |
| 1381 | accgtggaca agagcaggtg gcaggagggg aatgtcttct catgctccgt gatgcatgag |
| 1441 | gctctgcaca accactacac acagaagagc ctctccctgt ctctgggtaa aatcacgtgc |
| 1501 | cctcccccca tgtccgtgga acacgcagac atctgggtca agagctacag cttgtactcc |
| 1561 | agggagcggt acatttgtaa ctctggtttc aagcgtaaag ccggcacgtc cagcctgacg |
| 1621 | gagtgcgtgt tgaacaaggc cacgaatgtc gcccactgga caacccccag tctcaaatgc |
| 1681 | attagaagcg gcggcagcgg cggcggcggc agcggcggcg gcagcggcgg cggcggcagc |
| 1741 | ctgcagaact gggtgaatgt aataagtgat ttgaaaaaaa ttgaagatct tattcaatct |
| 1801 | atgcatattg atgctacttt atatacggaa agtgatgttc accccagttg caaagtaaca |
| 1861 | gcaatgaagt gctttctctt ggagttacaa gttatttcac ttgagtccgg agatgcaagt |
| 1921 | attcatgata cagtagaaaa tctgatcatc ctagcaaaca acagtttgtc ttctaatggg |
| 1981 | aatgtaacag aatctggatg caaagaatgt gaggaactgg aggaaaaaaa tattaaagaa |
| 2041 | tttttgcaga gttttgtaca tattgtccaa atgttcatca acacttct |

In some embodiments, the mabkine-encoding nucleic acid has the nucleic acid sequence set forth below (SEQ ID NO: 70), which encodings Mabkine #3 (SEQ ID NO: 66) that contains the elements set forth in Table 10.

| | |
|---|---|
| 1 | atgtacagga tgcaactcct gtcttgcatt gcactaagtc ttgcacttgt cacgaattcg |
| 61 | catcaccatc accatcacga catccagatg actcagtctc cagcctccct atctgcatct |
| 121 | gtgggagaaa ctgtcaccat cacatgtcga gcaagtgaga atatttacag ttatttagca |
| 181 | tggtatcagc agaaacaggg aaaatctcct cagttcttgg tctataatgc aaaaacctta |
| 241 | gcagaaggtg tgccatcaag gttcagtggc agtggatcag gcacacagtt ttctctgaag |
| 301 | atcaacagcc tacagcctga agattttggg agttattact gtcaacatca ctatggtact |
| 361 | cctcggacgt tcggtggagg caccaagctg gaaatcaaag gtggaggtgg cagcggagga |
| 421 | ggtgggtccg gcggtggagg aagccaggtc caactgcagc agcctggggc tgagctggtg |
| 481 | aggcctgggg cttcagtgaa gctgtcctgc aaggcttctg gctacacgtt caccagctac |
| 541 | tggatgaact gggttaagca gaggcctgag caaggccttc agtggattgg aaggattgat |
| 601 | ccttacgata gtgaaactca ctacagtcaa aagttcaagg acaaggccat attgactcta |
| 661 | gacaaatcct ccagcacagc ctacatgcga ctcagcagcc tgacatctga ggactctgcg |
| 721 | gtctattact gtgcaagagg gggctatgat ttcgacgtag gaactctcta ctggttcttc |
| 781 | gatgtctggg gcgcagggac cacggtcacc gtctcctcac ccccatgccc accgtgccca |
| 841 | gcacctgagt tcctgggggg accatcagtc ttcctgttcc ccccaaaacc caaggacact |
| 901 | ctcatgatct cccggacccc tgaggtcacg tgcgtggtgg tggacgtgag ccaggaagac |
| 961 | cccgaggtcc agttcaactg gtacgtggat ggcgtggagg tgcataatgc caagacaaag |
| 1021 | ccgcgggagg agcagttcaa cagcacgtac cgtgtggtca gcgtcctcac cgtcctgcac |
| 1081 | caggactggc tgaacggcaa ggagtacaag tgcaaggtct ccaacaaagg cctcccgtcc |
| 1141 | tccatcgaga aaaccatctc caaagccaaa gggcagcccc gagagccaca ggtgtacacc |
| 1201 | ctgcccccat cccaggagga gatgaccaag aaccaggtca gcctgacctg cctggtcaaa |
| 1261 | ggcttctacc ccagcgacat cgccgtggag tgggagagca atgggcagcc ggagaacaac |
| 1321 | tacaagacca cgcctcccgt gctggactcc gacggctcct tcttcctcta cagcaggcta |
| 1381 | accgtggaca agagcaggtg gcaggagggg aatgtcttct catgctccgt gatgcatgag |
| 1441 | gctctgcaca accactacac acagaagagc ctctccctgt ctctgggtaa aatcacgtgc |
| 1501 | cctcccccca tgtccgtgga acacgcagac atctgggtca agagctacag cttgtactcc |
| 1561 | agggagcggt acatttgtaa ctctggtttc aagcgtaaag ccggcacgtc cagcctgacg |
| 1621 | gagtgcgtgt tgaacaaggc cacgaatgtc gcccactgga caacccccag tctcaaatgc |
| 1681 | attagaagcg gcggcagcgg cggcggcggc agcggcggcg gcagcggcgg cggcggcagc |
| 1741 | ctgcagaact gggtgaatgt aataagtgat ttgaaaaaaa ttgaagatct tattcaatct |
| 1801 | atgcatattg atgctacttt atatacggaa agtgatgttc accccagttg caaagtaaca |
| 1861 | gcaatgaagt gctttctctt ggagttacaa gttatttcac ttgagtccgg agatgcaagt |
| 1921 | attcatgata cagtagaaaa tctgatcatc ctagcaaaca acagtttgtc ttctaatggg |
| 1981 | aatgtaacag aatctggatg caaagaatgt gaggaactgg aggaaaaaaa tattaaagaa |
| 2041 | tttttgcaga gttttgtaca tattgtccaa atgttcatca acacttct |

### Vectors

The mabkine-encoding nucleic acid may be introduced into a cell by a suitable vector. A vector is configured and contains the elements necessary to effect transport into the cell and effect expression of the nucleic acid(s) after transformation. Such elements include an origin of replication, a poly-A tail sequence, a selectable marker, and one or more suitable sites for the insertion of the nucleic acid sequences, such as a multiple cloning site (MCS), one or more suitable promoters, each promoter operatively linked to the insertion sites of the nucleic acid sequences and the selectable marker, and additional optional regulatory elements.

The term “promoter” as used herein refers to a nucleic acid sequence that regulates, directly or indirectly, the transcription of a corresponding nucleic acid coding sequence to which it is operably linked, which in the context of the present disclosure, is an mabkine. A promoter may function alone to regulate transcription, or it may act in concert with one or more other regulatory sequences (e.g., enhancers or silencers, or regulatory elements that may be present in the nucleic acid sequences or the vector). Promoters are located near the transcription start sites of genes, on the same strand and upstream on the DNA (towards the 5′ region of the sense strand). Promoters typically range from about 100-1000 base pairs in length.

The term “operatively linked” as used herein is to be understood that a nucleic acid sequence is spatially situated or disposed in the vector relative to another nucleic acid sequence, e.g., a promoter is operatively linked to drive the expression of a nucleic acid coding sequence (e.g., the mabkine-encoding nucleic acid sequence).

In some embodiments, the vector has a strong mammalian promoter, for example a cytomegalovirus (CMV) promoter, a simian virus 40 (SV40) early promoter, synthetic promoters (e.g., RPBSA (synthetic, from Sleeping Beauty), or CAG (synthetic, CMV early enhancer element, chicken β-Actin, and splice acceptor of rabbit β-Globin)) or promoters derived from the β-actin, phosphoglycerate kinase (PGK), or factor EF1α genes. In some embodiments, the promoter may have a core region located close to the nucleic acid coding sequence. In some embodiments, the promoter is modified to remove methylation sensitive motifs (e.g., a cytosine nucleotide is followed by a guanine nucleotide, or “CpG”), or by the addition of a regulatory sequence that binds transcriptional factors that repress DNA methylation. In some embodiments, the vector includes A/T-rich, nuclear matrix interacting sequences, known as scaffold matrix attachment regions (S/MAR), which enhance transformation efficiency and improve the stability of transgene expression.

In some embodiments, the vector is a viral vector, for example, a retroviral vector, a lentiviral vector, an adenoviral vector, a herpesvirus vector, an adenovirus, or an adeno-associated virus (AAV) vector. As used herein, the term “lentiviral vector” is intended to mean an infectious lentiviral particle. Lentivirinae (lentiviruses) is a subfamily of enveloped retrovirinae (retroviruses), that are distinguishable from other viruses by virion structure, host range, and pathological effects. An infectious lentiviral particle will be capable of invading a target host cell, including infecting, and transducing non-dividing cells and immune cells.

In some embodiments, the vector containing RNA is a non-integrative and non-replicative recombinant lentivirus vector. The construction of lentiviral vectors has been described, for example, in U.S. Pat. Nos. 5,665,577, 5,981,276, 6,013,516, 7,090,837, 8,119,119, and 10,954,530. Lentivirus vectors include a defective lentiviral genome, i.e., in which at least one of the lentivirus genes gag, pol, and env, has been inactivated or deleted.

In other embodiments, the vector is a non-viral vector, representative examples of which include plasmids, mRNA, linear single stranded (ss) DNA or linear double stranded (ds) DNA, minicircles, and transposon-based vectors, such as Sleeping Beauty (SB)-based vectors and piggyBac™ (PB)-based vectors. In yet other embodiments, the vector may include both viral and non-viral elements.

In some embodiments the vector is a plasmid. In addition to a promoter operatively linked to the nucleic acids, the plasmid may also contain other elements e.g., that facilitate transport and expression of the nucleic acid in an immune cell. The plasmid may be linearized with restriction enzymes, in vitro transcribed to produce mRNA, and then modified with a 5′ cap and 3′ poly-A tail. In some embodiments, the vector multiple plasmids, a first plasmid encoding the fusion protein and a second plasmid encoding the immunocytokine.

In some embodiments, the vector may be disposed (e.g., encapsulated) in a carrier. The carrier may be lipid-based, e.g., lipid nanoparticles (LNPs), liposomes, lipid vesicles, or lipoplexes. In some embodiments, the carrier is an LNP. In certain embodiments, an LNP includes two or more concentric bilayers separated by aqueous compartments. Lipid bilayers may be functionalized and/or crosslinked to one another. Lipid bilayers may include one or more ligands, proteins, or channels.

Lipid carriers, e.g., LNPs may include one or more cationic/ionizable lipids, one or more polymer conjugated lipids, one or more structural lipids, and/or one or more phospholipids. A “cationic lipid” refers to positively charged lipid or a lipid capable of holding a positive charge. Cationic lipids include one or more amine group(s) which bear the positive charge, depending on pH. A “polymer conjugated lipid” refers to a lipid with a conjugated polymer portion. Polymer conjugated lipids include a pegylated lipids, which are lipids conjugated to polyethylene glycol. A “structure lipid” refers to a non-cationic lipid that does not have a net charge at physiological pH. Exemplary structural lipids include cholesterol, fecosterol, sitosterol, ergosterol, campesterol and the like. A “phospholipid” refers to lipids that have a triester of glycerol with two fatty acids and one phosphate ion. Phospholipids in LNPs assemble the lipids into one or more lipid bilayers. LNPs, their method of preparation, formulation, and delivery are disclosed in, e.g., U.S. Patent Application Publication Nos. 2004/0142025, 2007/0042031, and 2020/0237679 and U.S. Pat. Nos. 9,364,435, 9,518,272, 10,022,435, and 11,191,849.

Lipoplexes, liposomes, and lipid nanoparticles may include a combination of lipid molecules, e.g., a cationic lipid, a neutral lipid, an anionic lipid, polypeptide-lipid conjugates, and other stabilization components. Representative stabilization components include antioxidants, surfactants, and salts. Compositions and preparation methods of lipoplexes, liposomes, and lipid nanoparticles are known in the art. See, e.g., U.S. Pat. Nos. 8,058,069, 8,969,353, 9,682,139, 10,238,754, U.S. Patent Application Publications 2005/0064026 and 2018/0291086, and Lasic, Trends Biotechnol. 16(7):307-21(1998), Lasic et al., FEBS Lett. 312(2-3):255-8(1992), and Drummond et al., Pharmacol. Rev. 51(4):691-743 (1999).

### Cells

One aspect of the present disclosure is a genetically modified (or transformed) cell containing a vector that contains a nucleic acid encoding the mabkine for the purpose of making and purifying the mabkine.

Cells useful for the cloning and other manipulations of these vectors are conventional. Cells from various strains of E. coli may be used for replication of the vectors and other steps in the construction of the mabkines of this disclosure.

Suitable host cells or cell lines for the expression of the nucleic acid-encoding mabkines include eukaryotic cells. In some embodiments, the cells are mammalian cells such as CHO (e.g., DG44, CHO-S), fibroblast cells (e.g., 3T3, COS), embryonic cells (e.g., PER.C6, HEK (e.g., HEK.293)), somatic cell hybrids (e.g., Sp2/0), and cancer cells, for example, myeloma cells (e.g., NS0). In some embodiments, the nucleic acids encoding the mabkine is expressed in a CHO or a myeloma cell. Human cells may be used, thus enabling the molecule to be modified with human glycosylation patterns. The selection of suitable mammalian cells and methods for transformation, culture, amplification, screening and product production and purification are known in the art. See, e.g., Green et al., eds., Molecular Cloning: A Laboratory Manual, 5^{th} ed., Cold Spring Harbor Laboratory Press, New York, 2012.

In some embodiments, the cells are prokaryotic. Prokaryotic (i.e., bacterial) cells may prove useful as host cells suitable for the expression of the nucleic acids encoding mabkines (see, e.g., Pluckthun, Immunol. Rev., 130:151-188 (1992)). However, due to the tendency of proteins expressed in bacterial cells to be in an unfolded or improperly folded form or in a non-glycosylated form, any mabkines produced in a bacterial cell would be screened for retention of function (e.g., anti-NKG2A binding ability). If the mabkine expressed by the bacterial cell was produced in a properly folded form, that bacterial cell would be a desirable host, or in alternative embodiments the mabkine may express in the bacterial host and then be subsequently re-folded. For example, various strains of E. Coli used for expression are well-known as host cells in the field of biotechnology. Various strains of B. Subtilis, Streptomyces, other bacilli and the like may also be employed in this method.

After expression in a cell, the mabkines are isolated from the cell (e.g., cell lysates) or from the medium in which the cell is cultured. Protein isolation techniques are known in the art. Representative isolation techniques include chromatography, affinity chromatography, high performance liquid chromatography (HPLC), hydroxylapatite chromatography, protein A-Sepharose, gel electrophoresis, and dialysis.

In some embodiments, the mabkine is isolated by passage through an affinity chromatography resin, typically in the presence of a neutral phosphate buffer. The affinity chromatography resin in then subjected to an acidic buffer with a pH of about 3 to about 4, washing mabkine off of the affinity chromatography resin. A basic buffer may be used to neutralize the acidic buffer, then a tangential flow filtration of the neutralized buffer can be performed with a formulation buffer, to isolate a concentrated and purified solution containing the mabkine. In some embodiments, the affinity chromatography resin is a Protein A affinity chromatography resin or a Protein G affinity chromatography resin.

Additional protein isolation systems and methods are known in the art. See, e.g., U.S. Pat. Nos. 516,9936, 6,267,958, 8,357,778, 9,630,165, 9,708,399, 10,023,608, 11,369,703, and 11,390,668, U.S. Patent Application Publications 2017/0158760, 2019/0276492, and 2021/0206815, and Traunecker et al., Embo J. 10(12):3655-9 (1991).

Another aspect of the present disclosure is a genetically modified (or transformed) immune cell containing a nucleic acid encoding the mabkine. In some embodiments, the genetically modified immune cell also contains a nucleic acid encoding a CAR. In cells containing both a nucleic acid encoding a mabkine and a nucleic acid encoding a CAR, these nucleic acids may be disposed in the same or different vectors. The CAR contains an extracellular domain that binds an antigen on a cancer cell, a transmembrane domain, and an intracellular domain comprising a stimulatory domain. The CAR may optionally contain a hinge domain and additional co-stimulatory domains.

As used herein, “immune cell” refers to a cell of hematopoietic origin functionally involved in the initiation and/or execution of innate and/or adaptative immune response. The immune cells that contain a nucleic acid encoding a mabkine are referred herein as “mabkine immune cells.” The immune cells that contain a nucleic acid encoding a CAR are referred herein as “CAR immune cells.” CAR immune cells may further contain a nucleic acid encoding a mabkine.

Representative examples of immune cells include natural killer (NK) cells, T cells, macrophages, and dendritic cells. Combination of different immune cells may be used. In some embodiments, the immune cells are NK cells. In some embodiments, the immune cells are a NK cell line, primary NK cells, memory-like NK cells, or induced memory like NK cells.

Representative examples of T cells include cytotoxic lymphocytes, cytotoxic T cells (CD8⁺ T cells), T helper cells (CD4⁺ T cells), αβ T cells and/or γδ T cells NK T (NKT) cells, and Th17 T-cells. In some embodiments, the immune cells are CD8⁺ T cells. In some embodiments, the immune cells are CD4⁺ T cells. In some embodiments, the immune cells are a combination of CD8⁺ T cells and CD4⁺ T cells. CAR T cells may be primary T cells isolated from healthy patients and engineered to express a mabkine and a CAR. In some embodiments, the immune cells are macrophages.

Administration of the mabkine immune cells and/or the CAR immune cells may be autologous or allogeneic. For example, immune cells or progenitors thereof can be isolated from a tissue of body fluid from one subject prior to administration to the same subject (autologous) or a different, compatible subject (allogeneic).

The extracellular domain of the CAR that binds the cancer antigen may contain an antibody fragment. In some embodiments, the CAR binds to BCMA. CAR extracellular domains that bind to BCMA are known in the art. See, e.g., FDA-approved CAR-expressing immune cells ciltacabtagene autoleucel (Carvykti®), and idecabtagene vicleucel (Abecma®), U.S. Pat. Nos. 10,072,088, 10,683,369, 11,084,880, and 10,174,095, and U.S. Patent Application Publications 2016/0131655, 2017/0226216, 2018/0133296, 2019/0151365, 2019/0359727, 2019/0381171, 2020/0339699, 2020/0360431, 2020/0055948, and 2022/0064316. In some embodiments, the CAR extracellular domain is derived from a commercially available anti-BCMA antibody, BCMA-binding fragment, or derivative thereof, e.g., belantamab (Blenrep®), linvoseltamab (REGN5458), pacanalotamab (AMG 420), pavurutamab (AMG 701), and teclistamab (Tecvayli®).

In some embodiments, the CAR binds CD19. CAR extracellular domains that bind to CD19 are known in the art. See, e.g., FDA-approved CAR-expressing immune cells lisocabtagene maraleucel (Breyanzi®), tisagenlecleucel (Kymriah®), brexucabtagene autoleucel (Tecartus®), and axicabtagene ciloleucel (Yescarta®), U.S. Pat. Nos. 9,629,877, 10,273,300, and 10,533,055, U.S. Patent Application Publications 2020/0392248, and 2021/0238253, and Liu et al., N. Engl. J. Med. 382(6):545-553 (2020). In some embodiments, the CAR extracellular domain is derived from a commercially available anti-CD19 antibody, anti-CD19-binding fragment, or derivative thereof, e.g., loncastuximab (Zynlonta®), tafasitamab (Monjuvi®), denintuzumab (SGN-CD19A), and inebilizumab (Uplizna®).

In some embodiments, the CAR binds CD20. CAR extracellular domains that bind to CD20 are known in the art. See, e.g., U.S. Pat. Nos. 10,189,903, 10,442,867, 10,934,363, 11,066,457, 11,160,833, and 11,439,665, and U.S. Patent Application Publication 2018/0187149. In some embodiments, the CAR extracellular domain is derived from a commercially available anti-CD20 antibody, anti-CD20-binding fragment, or derivatives thereof, e.g., ofatumumab (Arzerra®, Kesimpta®), veltuzumab (IMMU-106), tositumomab (Bexxar®), and rituximab (Rituxan®, Riabni®, Truximab®).

In some embodiments, the CAR binds CD22. CAR extracellular domains that bind to CD22 are known in the art. See, e.g., U.S. Pat. Nos. 9,139,649, 9,181,343, and 10,494,435, U.S. Patent Application Publications 2015/0175711, 2018/0086843, 2021/0047402, 2021/0095022, 2022/0220198, and 2022/0273710, and Fry et al., Nat. Med. 24(1):20-28 (2018). In some embodiments, the CAR extracellular domain is derived from a commercially available anti-CD22 antibody, anti-CD22-binding fragment, or derivatives thereof, e.g., bectumomab, epratuzumab, inotuzumab, moxetumomab, and epratuzumab.

In some embodiments, the CAR binds CD33. CAR extracellular domains that bind to CD33 are known in the art. See, e.g., U.S. Pat. Nos. 9,944,702 and 10,912,799, U.S. Patent Application Publications 2018/0187149, 2019/0135894, 2021/0030793, and 2021/0338729, and Tang et al., Am. J. Cancer Res. 8(6):1083-1089 (2018). In some embodiments, the CAR extracellular domain is derived from a commercially available anti-CD33 antibody, anti-CD33-binding fragment, or derivative thereof, e.g., eluvixtamab, emerfetamab, gemtuzumab ozogamicin, and vadastuximab.

In some embodiments, the CAR binds PD-L1. CAR extracellular domains that bind to PD-L1 are known in the art. In some embodiments, the CAR extracellular domain is derived from a commercially available anti-PD-L1 antibody, anti-PD-L1-binding fragment, or derivative thereof, e.g., atezolizumab (Tecentriq®), avelumab (Bavencio®), or durvalumab (Imfinzi®).

In some embodiments, the CAR binds HER2. CAR extracellular domains that bind to HER2 are known in the art. See, e.g., U.S. Pat. Nos. 9,574,014, and 10,918,705, U.S. Patent Application Publications 2022/0265796, 2020/0277400, 2020/0399397, 2021/0253729, and 2022/0089750, and Uherek et al., Blood 100(4):1265-73 (2002), Chmielewski et al., J. Immunol. 173(12):7647-53 (2004), Turatti et al., J. Immunother. 30(7):684-93 (2007), Kruschinski et al., Proc. Natl. Acad. Sci. U.S.A 105(45):17481-6(2008), Ahmed et al., Clin. Cancer Res. 16(2):474-85 (2010), Hegde et al., Mol. Ther. 21(11):2087-101 (2013), Alsamah et al., Int. J. Pharm. Clin. Res. 6(1):97-100 (2014), Schönfeld et al., Mol. Ther. 23(2):330-8 (2015), and Zhang et al., J. Natl. Cancer Inst. 109(2):djw217-11 (2016). In some embodiments, the CAR extracellular domain is derived from a commercially available anti-HER2 antibody, anti-HER2-binding fragment, or derivative thereof, e.g., margetuximab (Margenza®), pertuzumab (Perjeta®), runimotamab, trastuzumab (Herceptin®, Herzuma®, Kanjinti®, Ogivri®, Ontruzant®), zanidatamab, and zenocutuzumab.

In some embodiments, the CAR binds SLAMF7. CAR extracellular domains that bind to SLAMF7 are known in the art. See, e.g., U.S. Pat. No. 10,799,536, U.S. Patent Application Publications 2020/0024342, 2020/0283534, 2021/0230548, and 2021/0253729, and Chu et al., Leukemia 28(4):917-27 (2014). In some embodiments, the CAR extracellular domain is derived from a commercially available anti-SLAMF7 antibody, anti-SLAMF7-binding fragment, or derivative thereof, e.g., elotuzumab (Empliciti®).

The transmembrane domain of the CAR connects the CAR extracellular domain to the intracellular domain. In some embodiments, the transmembrane domain is directly connected to the CAR extracellular domain. Amino acid sequences of representative transmembrane domains are listed in Table 11.

TABLE 11
Amino Acid Sequences of Representative
Transmembrane Domains

| Transmembrane domain | Sequence |
|---|---|
| CD3ζ (SEQ ID NO: 71) | LCYLLDGILFIYGVILTALFL |
| CD4 (SEQ ID NO: 72) | MALIVLGGVAGLLLFIGLGIFF |
| CD8α (SEQ ID NO: 73) | IYIWAPLAGTCGVLLLSLVITLYC |
| CD28 (SEQ ID NO: 74) | FWVLVVVGGVLACYSLLVTVAFIIFWV |
| CD137 (4-1BB) | IISFFLALTSTALLFLLFFLTLRFSVV |
| (SEQ ID NO: 75) | |

The amino acid sequence of a naturally occurring transmembrane domain may be modified by an amino acid substitution to avoid binding of such regions to the transmembrane domain of the same or different surface membrane proteins to minimize interactions with other members of a receptor complex. See, e.g., U.S. Patent Application Publication 2021/0101954; Soudais et al., Nat. Genet. 3:77-81 (1993); Muller et al., Front. Immunol. 12:639818-13 (2021); Elazar et al., elife 11:e75660-29 (2022).

In some embodiments, the CAR includes a hinge domain disposed between the CAR extracellular domain and the transmembrane domain. A hinge domain may provide flexibility in terms of allowing the CAR extracellular domain to obtain an optimal orientation for antigen-binding, enhancing antitumor activities of the cell expressing the CAR. Amino acid sequences of representative hinge domains are listed in Table 5.

The intracellular domain of the CAR further contains a signaling domain that enables intracellular signaling and immune cell function. The signaling domain may include a primary signaling domain and/or a co-stimulatory signaling domain. In some embodiments, the intracellular domain is capable of delivering a signal approximating that of natural ligation of an ITAM-containing molecule or receptor complex such as a TCR receptor complex.

In some embodiments, the signaling domain includes a plurality, e.g., 2 or 3, costimulatory signaling domains, e.g., selected from 4-1BB, CD3ζ, CD28, CD27, ICOS, and OX40. In some embodiments, the signaling domain may include a CD3ζ domain as a primary signaling domain, and any of the following pairs of co-stimulatory signaling domains from the extracellular to the intracellular direction: 4-1BB-CD27; CD27-4-1BB; 4-1BB-CD28; CD28-4-1BB; OX40-CD28; CD28-OX40; 4-1BB-CD3ζ; CD3ζ-4-1BB; CD28-CD32; CD32-CD28; CD28-4-1BB and 4-1BB-CD28. In some embodiments the primary signaling domain is derived from CD35, CD27, CD28, CD40, KIR2DS2, MyD88, or OX40. In some embodiments, the co-stimulatory signaling domain is derived from one or more of CD3γ, CD3δ, CD3ε, CD3ζ, CD4, CD5, CD8α, CD9, CD16, CD22, CD27, CD28, CD33, CD37, CD40, CD45, CD68, CD72, CD80, CD86, CD137 (4-1BB; TNFRSF9), CD154, CLEC-1, 4-1BB, DAP10 (hematopoietic cell signal transducer ((HCST)), DAP12 (TYROBP), Dectin-1, FcαRI, FcγRI, FcγRII, FcγRIII, IL-2RB, ICOS, KIR2DS2, MyD88, OX40, and ZAP70.

A representative CAR with a CD3ζ stimulatory signaling domain is the FDA-approved CAR-expressing immune cells tisagenlecleucel (Kymriah®). Representative CARs with CD3ζ and 4-1BB co-stimulatory signaling domains are the FDA-approved CAR-expressing immune cells idecabtagene vicleucel (Abecma®), lisocabtagene maraleucel (Breyanzi®), and ciltacabtagene autoleucel (Carvykti®). Representative CARs with CD28 and CD3ζ co-stimulatory signaling domains are the FDA-approved CAR-expressing immune cells brexucabtagene autoleucel (Tecartus®) and axicabtagene ciloleucel (Yescarta®).

In some embodiments, the CAR immune cell is a NK cell. In some embodiments, the CAR immune cell is a T cell. Additional CAR immune cells are known in the art, e.g., U.S. Pat. Nos. 5,906,936, 7,446,190, 7,741,465, 8,389,282, 8,399,645, 9,422,351, 9,790,267, 9,885,298, 10,124,023, 10,815,301, and 11,433,100 and U.S. Patent Application Publications 2019/0375815, 2020/0281973, 2021/0300986, 2022/0056101, and 2022/0193138.

### Pharmaceutical Compositions

Pharmaceutical compositions of the disclosure contain an effective amount of the mabkine and a pharmaceutically acceptable carrier. The term “effective amount” as used herein refers to a sufficient amount of mabkine to provide the desired effect of activating immune cells as measured by an increase of immune cell activation markers. The amount of mabkine administered to a subject will vary between wide limits, depending upon the location, type, and severity of the cancer, the age, body weight, and condition of the individual to be treated, etc. A physician will ultimately determine appropriate doses to be used.

The amount of mabkine administered to a subject may vary between wide limits, depending upon numerous factors such as the location, type, and severity of the cancer, and the age, body weight, and condition of the individual to be treated. A physician will ultimately determine appropriate amount of mabkine and doses to be used. Typically, the mabkine will be administered in a series of doses. In some embodiments, the effective amount of the mabkine is between approximately 50 to approximately 180 mg per subject per dose. In some embodiments, the effective amount of the mabkine is between approximately 1 to approximately 18 mg per kg of subject body weight.

In a related aspect, the disclosure provides a pharmaceutical composition that contains an effective number of immune cells that contain a nucleic acid encoding a mabkine, and a pharmaceutically acceptable carrier. The term “effective number of immune cells” (which indirectly includes a corresponding amount of a mabkine) as used herein refers to a sufficient number of the immune cells to provide the desired effect. In some embodiments, the immune cells contain both (i) a nucleic acid encoding a mabkine and (ii) a nucleic acid encoding a CAR.

In some embodiments, the pharmaceutical composition further contains an effective number of CAR immune cells that contain a nucleic acid encoding a mabkine, and a pharmaceutically acceptable carrier.

The number of immune cells administered to a subject will vary between wide limits, depending upon the location, type, and severity of the cancer, the age, body weight, and condition of the individual to be treated, and etc. A physician will ultimately determine appropriate number of cells and doses to be used. Typically, the CAR immune cells will be given in a single dose. In some embodiments, the effective number of the CAR immune cells is between approximately 1×10⁵ to approximately 1×10¹⁰ cells per subject. In some embodiments, the effective number of the CAR immune cells is between approximately 1×10⁵ to approximately 6×10⁸ cells per kg of subject body weight.

Compositions may be provided as sterile solid or liquid preparations. Solid preparations may be reconstituted and diluted into a liquid preparation before use, e.g., with carriers containing isotonic aqueous solutions, suspensions, emulsions, dispersions, or viscous solutions, which may be buffered to a selected pH. Liquid, pharmaceutically acceptable carriers include aqueous or non-aqueous carriers alike. Representative examples of liquid carriers include sterile water for injection, saline, Lactated Ringer Injection solution, phosphate buffered saline, a soluble protein, soluble sugars (e.g., dextrose), dimethyl sulfoxide (DMSO), polyol (e.g., glycerol, propylene glycol, liquid polyethylene glycol, and the like), ethanol, and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Citric acid, sodium chloride, sugars, polyalcohols, and isotonic electrolyte solutions (e.g., Plasma-Lyte®) may be used to achieve the desired isotonicity. Depending on the carrier and the immunocytokine, other excipients may be added, e.g., wetting, dispersing, or emulsifying agents, gelling and viscosity enhancing agents, preservatives and the like as known in the art. In some embodiments, the compositions include citric acid, ethylenediaminetetraacetic acid (EDTA), and polysorbate 20 with a pH range between about 6.8 to about 7.2.

### Methods of Use

In some aspects, the present disclosure is directed to treating a cancer in a subject. The method entails administering to a subject in need thereof a pharmaceutical composition containing an mabkine described herein. The term “cancer” as used herein refers to a disease or disorder characterized by excess proliferation or reduced apoptosis in a subject. Cancers that may be treated with the mabkines disclosed herein include both hematopoietic cancers and cancers characterized by the presence of a solid tumor.

The term “subject” (or “patient”) as used herein includes all members of the animal kingdom prone (or disposed) to or suffering from the indicated cancer. In some embodiments, the subject is a human. Therefore, a subject “having a cancer” or “in need of” treatment according to the present disclosure broadly embraces subjects who have been positively diagnosed, including subjects having active disease who may have been previously treated with one or more rounds of therapy, and subjects who are not currently being treated (e.g., in remission) but who might still be at risk of relapse, and subjects who have not been positively diagnosed but who are predisposed to a cancer (e.g., on account of the basis of prior medical history and/or family medical history, or who otherwise present with a one or more risk factors such that a medical professional might reasonably suspect that the subject was predisposed to cancer).

The terms “treat”, “treating”, and “treatment” as used herein refer to any type of intervention, process performed on, or the administration of an active agent to the subject in need thereof with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer.

In some embodiments, the cancer is a hematopoietic cancer. Representative hematological cancers include plasma cell neoplasm (e.g., myeloma, multiple myeloma, relapsed or refractory multiple myeloma, plasma cell myeloma, extramedullary multiple myeloma, monoclonal gammopathy of unknown significance (MUGS), asymptomatic smoldering multiple myeloma, or solitary plasmacytoma), lymphoma (e.g., Hodgkin's lymphoma, non-Hodgkin's lymphoma, Burkitt's lymphoma, plasmablastic lymphoma, plasmacytoid lymphoma, or diffuse large B-cell lymphoma), leukemia (e.g., relapsed or refractory acute B lymphocytic leukemia, or relapsed or refractory acute lymphoblastic leukemia), and carcinomas (e.g., Waldenstrom macroglobulinemia or glioblastoma (astrocytoma)). In these embodiments, the therapeutic effect might include on or more art-recognized indicia of therapeutic efficacy, representative examples of which include prevention or prolongation of metastases, improvement in survival time, total/complete or partial remission of a cancer, e.g., no detectable cancer cells and less tumor cells or smaller tumors, respectively, or a reduction in tumor cell number. In some embodiments, the hematopoietic cancer is multiple myeloma, lymphoma, or leukemia.

In some embodiments, the cancer is characterized by the presence of a solid tumor. In some embodiments, the cancer is a bladder cancer (e.g., transitional cell carcinoma, also called urothelial carcinoma), kidney cancer (e.g., renal cell carcinoma (RCC), kidney renal clear cell carcinoma (KIRC), transitional cell cancer, or Wilms tumor), skin cancer (e.g., melanoma, skin cutaneous melanoma (SKCM), basal cell carcinoma, and squamous cell carcinoma of the skin), lung cancer (e.g., small cell lung cancer, non-small cell lung cancer, including lung adenocarcinoma (LUAD) and lung squamous cell carcinoma (LUSC)), head and neck cancer (e.g., squamous cell carcinoma of the head and neck (SCCHN) also called head and neck squamous cell carcinoma (HNSC), laryngeal and hypopharyngeal cancer, nasal cavity and paranasal sinus cancer, nasopharyngeal cancer, oral and oropharyngeal cancer, and salivary gland cancer), colon or rectal cancer (e.g., colorectal carcinoma (CRC), colon adenocarcinoma (COAD), rectum adenocarcinoma (READ)), ovarian cancer (e.g., cystadenocarcinoma, ovarian embryonal carcinoma, ovarian adenocarcinoma, epithelial ovarian carcinomas, fallopian tube cancer, and primary peritoneal cancer), endometrial cancer, cervical cancer (e.g., cervical squamous cell carcinoma and endocervical adenocarcinoma (CESC)), prostate cancer (e.g., prostate adenocarcinoma (PRAD)), and stomach cancer (e.g., stomach adenocarcinoma (STAD)).

### Administration

Pharmaceutical compositions of the present disclosure (which embrace the Mabkine, per se, and genetically modified immune cells that contain a nucleic acid that encodes the Mabkine) may be administered to a subject for the treatment of a cancer by any medically acceptable route. They are typically delivered intravenously, although they may also be introduced into other convenient sites (e.g., to an affected organ or tissue) or modes, as determined by an attending physician. In some embodiments, the pharmaceutical compositions are administered parenterally. In some embodiments, the pharmaceutical compositions are administered intratumorally (i.e., into a solid tumor).

In some embodiments, the composition is administered cyclically, for example, administered once a week, once every two weeks, once every three weeks. The cycle is repeated, for example, for 2 cycles, 3 cycles, 5 cycles, 8 cycles. In some embodiments, the mabkine is administered for a period of consecutive days before cyclic administration, for example, administered once a day for five days and once every three weeks thereafter. In some embodiments, the mabkine are administered as an intravenous infusion over a period of time. Representative infusion times are 30 minutes, 60 minutes, and 90 minutes. In some embodiments, the infusion time is between 30 and 60 minutes. In some embodiments, the first administration is infused into a patient for 90 minutes and administrations are infused into a patient for 30 minutes.

In some embodiments, the composition is administered once every 3 weeks (a 21-day cycle) as an infusion over about 30 to about 90 minutes. In some embodiments, the mabkine is administered for 5 consecutive days every 3 weeks (21-day cycle) and repeated for 8 cycles.

The mabkine immune cells and/or the CAR immune cells are typically delivered once, e.g., intravenously. They may also be introduced into other convenient sites (e.g., to an affected organ or tissue) modes or administration cycles, as determined by an attending physician. In some embodiments, the method entails administering the CAR immune cells prior to the administration of the mabkine.

### Combination Therapy

In some embodiments, the present methods may include co-administration of another anti-cancer agent. The terms “co-administration,” “co-administer,” and “co-administered” include substantially contemporaneous administration, by the same or separate dosage forms, or sequentially, e.g., as part of the same treatment regimen or by way of successive treatment regimens. Thus, if given sequentially, at the onset of administration of the second therapy, the first of the two therapies is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

Anti-cancer agents that may be used in combination with the mabkines are known in the art. See, e.g., U.S. Pat. No. 9,101,622 (Section 5.2 thereof). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of cancerous cells. This process may involve contacting the cancer cells with recipient cells and the agent(s) or multiple factor(s) at the same time. This may be achieved by contacting the cancer cells with a single composition or pharmacological formulation that includes both agents, or by contacting the cancer cells with two distinct compositions or formulations, at the same time, wherein one composition includes recipient cells and the other includes the second agent(s).

In some embodiments, the mabkines of the present disclosure are used in conjunction with chemotherapeutic, radiotherapeutic, immunotherapeutic intervention, targeted therapy, pro-apoptotic therapy, or cell cycle regulation therapy.

### Immunotherapy

Immunotherapy, including co-administration of immune checkpoint inhibitors may be employed to treat a cancer. Immune checkpoint molecules include, for example, PD1, CTLA4, KIR, TIGIT, TIM-3, LAG-3, BTLA, VISTA, CD47, and NKG2A. Clinically available examples of immune checkpoint inhibitors include durvalumab (Imfinzi®), atezolizumab (Tecentriq®), and avelumab (Bavencio®). Clinically available examples of PD1 inhibitors include nivolumab (Opdivo®), pembrolizumab (Keytruda®), and cemiplimab (Libtayo®).

### Chemotherapy

Anti-cancer therapies also include a variety of combination therapies with both chemical and radiation-based treatments. Combination chemotherapies include, for example, Abraxane®, altretamine, docetaxel, Herceptin®, methotrexate, Novantrone®, Zoladex®, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, Taxol®, gemcitabien, Navelbine®, farnesyl-protein tansferase inhibitors, transplatinum, 5-fluorouracil, vincristine, vinblastine and methotrexate, or any analog or derivative variant of the foregoing and also combinations thereof.

### Radiotherapy

Anti-cancer therapies also include radiation-based, DNA-damaging treatments. Combination radiotherapies include what are commonly known as gamma-rays, X-rays, and/or the directed delivery of radioisotopes to tumor cells which cause a broad range of damage on DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells and will be determined by the attending physician.

Radiotherapy may include external or internal radiation therapy. External radiation therapy involves a radiation source outside the subject's body and sending the radiation toward the area of the cancer within the body. Internal radiation therapy uses a radioactive substance sealed in needles, seeds, wires, or catheters that are placed directly into or near the cancer.

These and other aspects of the present disclosure will be further appreciated upon consideration of the following working examples, which are intended to illustrate certain embodiments of the disclosure but are not intended to limit its scope, as defined by the claims.

### EXAMPLES

### Example 1: Materials and Methods

Vector design. As disclosed above, Mabkine #1 contains an IL-2 signal peptide, anti-NKG2A scFv, IgG4 hinge, IgG4 Fc domain, IL-15Ra sushi domain, linker, and IL-15 and has the amino acid sequence set forth in SEQ ID NO: 64; Mabkine #2 contains a His tag, an IL-2 signal peptide, anti-NKG2A scFv, IgG4 hinge, IgG4 Fc domain, IL-15Ra sushi domain, linker, and IL-15 and has the amino acid sequence set forth in SEQ ID NO: 65; and Mabkine #3 contains a His Tag, IL-2 signal peptide, anti-NKG2A scFv, IgG1 hinge, IL-15Ra sushi domain, linker and IL-15 and has the amino acid sequence set forth in SEQ ID NO: 66. Gene fragments encoding the mabkines were synthesized and cloned into a pUC57 vector (GenScript) and subcloned into UCOE plasmid via restriction enzyme cloning using FseI and BmtI.

The GpHLA-E contains the human leukocyte antigen (HLA)-G signal peptide, and human HLA-E ectodomain, transmembrane domain, and endodomain. A gene fragment encoding GpHLA-E was synthesized (Integrated DNA Technologies) and cloned into a pHIV vector to generate pHIV-GpHLA-E.

The pHIV-CD19-CAR-GFP plasmid was obtained from Dr. Jianzhu Chen (Massachusetts Institute of Technology). The CD19-CAR contains the CD8 signal peptide, an anti-CD19 scFv, CD8 hinge, CD8 transmembrane domain, 41BB intracellular co-signaling domain and CD3ζ intracellular co-signaling domain. Gene fragments encoding anti-HER2 scFv were synthesized (Integrated DNA Technologies) and cloned into pHIV-CD19-CAR-GFP plasmid by replacing the anti-CD19 scFv to generate pHIV-HER2-CAR-GFP. The anti-HER2 scFv was generated using codon-optimized DNA sequences of the VL and VH regions of trastuzumab.

Mabkine production. Mabkines were produced by transfecting CHO-S cells with mabkine plasmids using Lipofectamine™ 2000 (Invitrogen) and selected for transfection with antibiotics for 23 days. Culture supernatants (polyclones) were collected at 13 days post-transfection and mabkines were purified using protein A-sepharose beads resin, Eshmuno® A (Merck Cat. #1.20089) packed in a HiScale™ XK 16 column (Cytiva) with 16.5 cm height and a column volume of 40 mL. The purified mabkines were resuspended in (a) 25 mM phosphate buffered solution at pH 6-8 and stabilized with 137 mM NaCl or (b) 25 mM phosphate buffered solution at pH 6-7 and stabilized with 29 mM saccharose, 25 mM L-Arginine and 100 mM NaCl. The purified mabkines were stored at 2-8° C. or at −20° C.

Cell lines. Ramos and SKBR3 cell lines were obtained from the American Type Culture Collection (ATCC). Ramos cells were cultured in RP-10 medium containing RPMI 1640 supplemented with 10% FBS, 1× penicillin/streptomycin, 2 mM L-glutamine, and 7.5 mmol HEPES. SKBR3 cells were cultured in DMEM/F12 medium (Gibco) supplemented with 10% FBS and 1× penicillin/streptomycin. Ramos and SKBR3 cells were transduced with VSVG-pseudotyped lentivirus encoding GpHLA-E and/or mCherry-luciferase.

Generation and transduction of human primary NK cells. Human primary NK cells were isolated from leukoreduction system chamber from a healthy donor after incubation with RosetteSep™ human NK cell enrichment cocktail (StemCell) followed by Ficoll® density gradient centrifugation. Cells were stimulated for 18 hours with 10 ng/ml recombinant human IL-12 (R&D Systems) and 50 ng/mL recombinant human IL-18 (R&D Systems) in RP-10. NK cells were transduced with BaEV-pseudotyped lentivirus encoding CD19-CAR or HER2-CAR on RetroNectin™-coated plates (Takara Bio) with the addition of Vectofusin-1® (Miltenyi). After spinfection, cells were rested for 2 days in RP-10 with 1 ng/mL recombinant human IL-15 (Miltenyi). NK cells were then expanded in NK MACS medium (Miltenyi) supplemented with 5% human serum and 100 U/mL recombinant human IL-2 (Miltenyi).

Cytotoxicity of NK cells in vitro and intracellular cytokine staining. Ramos and SKBR3 target cells were labelled with 5 μM of CellTrace™ Violet (Thermo Fisher Scientific) in PBS for 20 min at 37° C. and washed twice with culture media. SKBR3 cells were labelled one day prior to co-culture and seeded at 25,000 cells per well in 96-well flat-bottom plate. Ramos cells were labelled on the same day of co-culture and seeded at 25,000 cells per well in 96-well U-bottom plate. NK cells were washed twice with RP-10 and co-cultured with target cells at the E:T ratios indicated in each experiment. To measure NK cell cytotoxicity, Ramos and SKBR3 cells were co-cultured with NK cells for 4 h and 24 h, respectively, then stained with 2 μL of PE-Annexin V (Biolegend) and 2 μL of 7-AAD (BD Biosciences) in 50 μL Annexin V binding buffer (Biolegend) for 15 min at room temperature (RT).

To measure intracellular IFNγ and degranulation, target cells and NK cells were co-cultured for 1 h, followed by the addition of 0.2 μL BD GolgiPlug™ (BD Biosciences), 0.13 μL BD GolgiStop™ (BD Biosciences) and 1 μL of APC-CD107a (Biolegend). After an additional 5 h of co-culture, NK cells were stained for intracellular IFNγ using BD Cytofix/Cytoperm™ (BD Biosciences). Cells were acquired using BD LSRFortessa™ and analyzed using FlowJo™ (Tree Star).

NKG2A occupancy and pSTAT5 staining. Primary NK cells were washed twice with RP-10, seeded at 50,000 cells per well in 96-well flat-bottom plate, rested for 30 min in the absence of cytokines, then stimulated with different concentrations of IL-15 or mabkine for 30 min. NK cells were then stained for surface expression of NKG2A. NK cells were also fixed with 100% methanol (pre-cooled at −20° C.) for 15 min on ice and stained for intracellular expression of pSTAT5.

Flow cytometry. Cells were stained with the following antibodies: anti-CD56 (HCD56) and anti-NKG2A (S19004C) from Biolegend, and anti-pSTAT5 (SRBCZX) from Invitrogen Fixable Yellow Live/Dead stain was from Invitrogen. To measure NK cell cytotoxicity, NK cells and target cells were co-cultured for 4 h, then stained with PE-Annexin V and 7-aminoactinomycin D (7-AAD) (Biolegend) for 15 min at RT. Cells were acquired using BD LSRFortessa™ and analyzed using FlowJo™ (Tree Star).

### Example 2: Mabkine Design, Cloning, and Expression

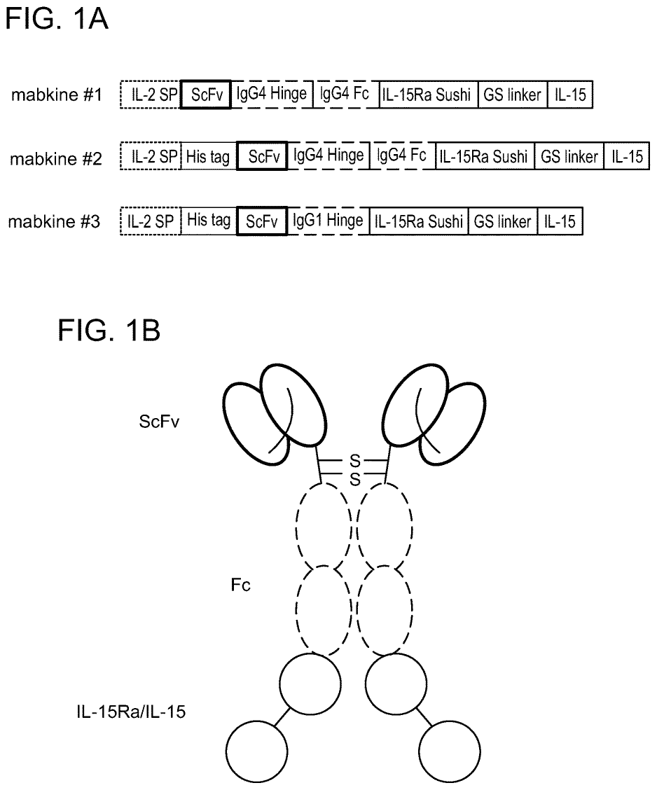

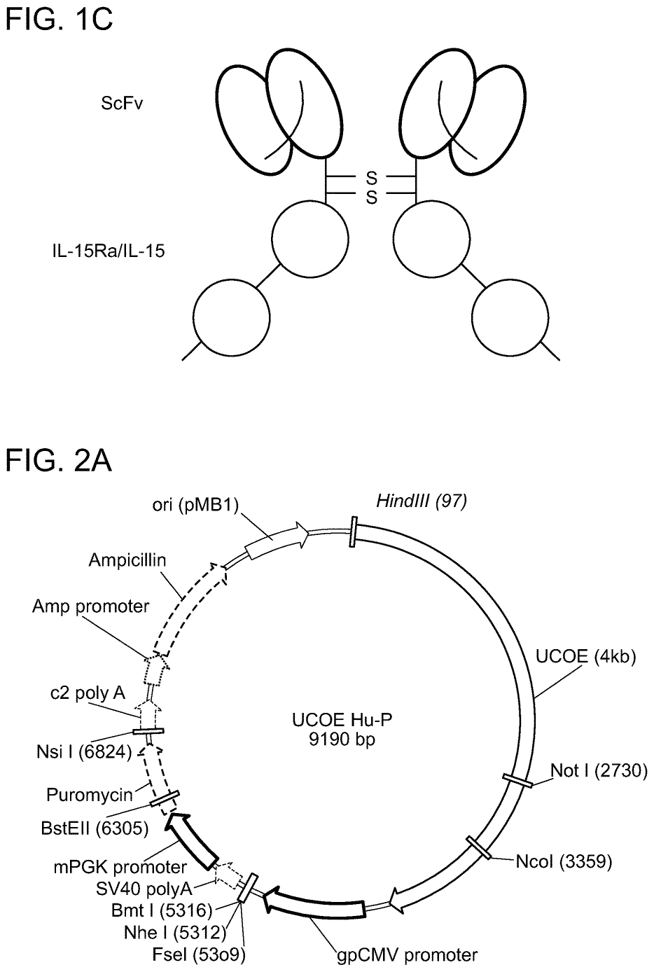

Three mabkines were designed, referred herein as Mabkine #1, Mabkine #2, and Mabkine #3 (FIG. 1A). Mabkine #1, schematically illustrated in FIG. 1B, contains an IL-2 signal peptide (IL-2 SP), a scFv that binds NKG2A (ScFv), a hinge domain derived from IgG4 (IgG4 hinge), an Fc region derived from IgG4 (IgG4 Fc), an IL-15Rα Sushi domain, a linker (GS linker), and an IL-15 domain. Mabkine #2, schematically illustrated in FIG. 1B, contains an IL-2 signal peptide, a selection marker (His tag) a scFv that binds NKG2A, a hinge domain derived from IgG4, an Fc region derived from IgG4, an IL-15Rα Sushi domain, a linker, and an IL-15 domain. Mabkine #3, schematically illustrated in FIG. 1C, contains an IL-2 signal peptide, a selection marker, a scFv that binds NKG2A, a hinge domain derived from IgG1, an IL-15Rα Sushi domain, a linker, and an IL-15 domain.

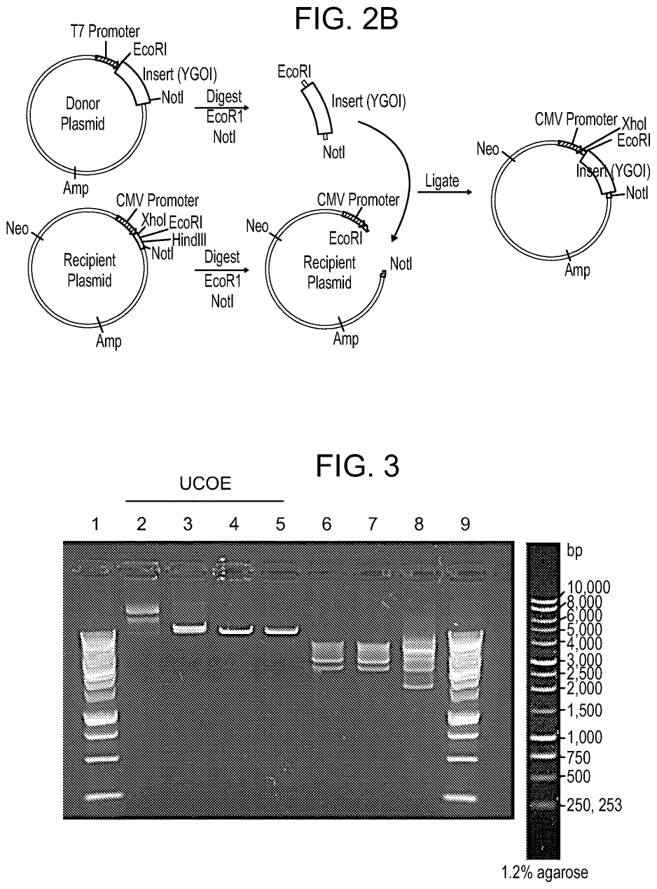

Mabkines-encoding nucleic acids may be incorporated into many different vectors. In this case, the mabkines were synthesized by the GenScript Corporation and cloned into the pUC57 or pCC1-4k vector donor plasmids. The mabkines were then incorporated into commercially available UCOE® Human 4 kb Purpo Set, as illustrated in FIG. 2A. The methodology for cloning of the mabkine-encoding nucleic acid was conducted as illustrated in FIG. 2B, and described as follows. The donor vectors (pUC57 and pCC1-4k) are suitable for vector replication in bacteria and were digested (cut) on both ends of the mabkine-encoding nucleic acid with the restriction enzymes FseI and BmtI. The recipient vector (UCOE®) was similarly digested. The insert was then inserted into the recipient vector via a ligation step. The resulting vectors were run on a gel to confirm the size of each DNA fragment (FIG. 3). The lanes illustrated in FIG. 3 were loaded with ladder in lane 1, UCOE® vector control (without an mabkine-encoding nucleic acid) in lanes 2-5, and lanes 6-8 received the UCOE® vector with an mabkine-encoding nucleic acid digested with both FseI and BmtI digestion. Lane 2 received UCOE® vector control without digestion, lane 3 received UCOE® vector control with FseI digestion, lane 4 received UCOE® vector control with BmtI digestion, lane 5 received UCOE® vector control with FseI and BmtI digestion. Lane 6 received UCOE® vector containing Mabkine #1, lane 7 received UCOE® vector containing Mabkine #2, and lane 8 received UCOE® vector containing Mabkine #3.

### Example 3: NKG2A-Binding Mabkines Bind Primary NK Cells

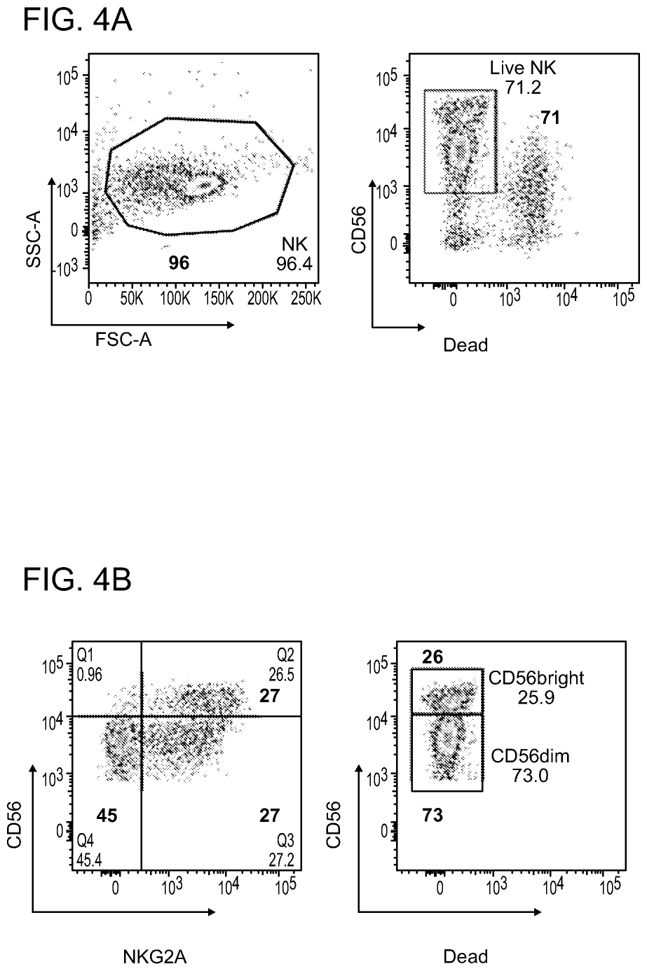

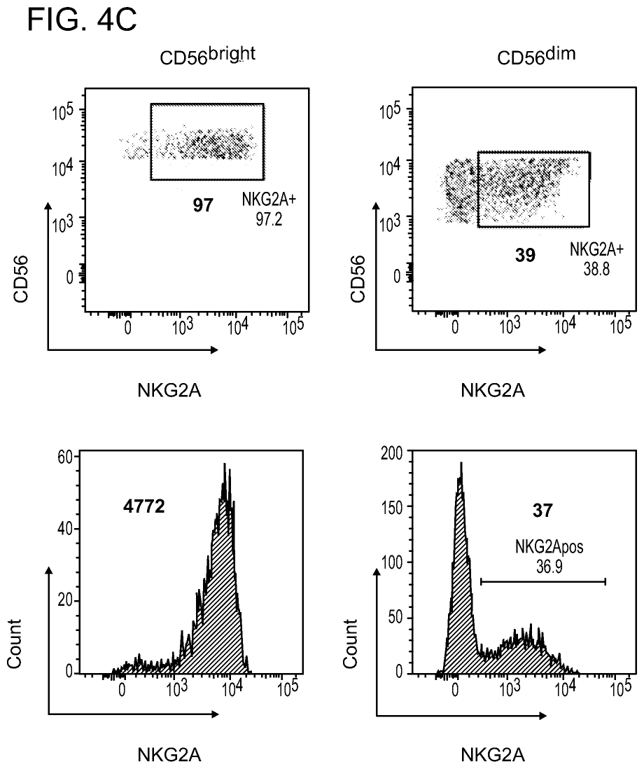

Different subsets of primary NK cells express NKG2A. Peripheral blood NK cells (primary NK cells) were detected with anti-CD56 antibody and assayed for NKG2A expression (FIG. 4A-FIG. 4C). 97% of CD56^{bright} NK cells expressed NKG2A, and 39% of CD56^{dim} NK cells expressed NKG2A.

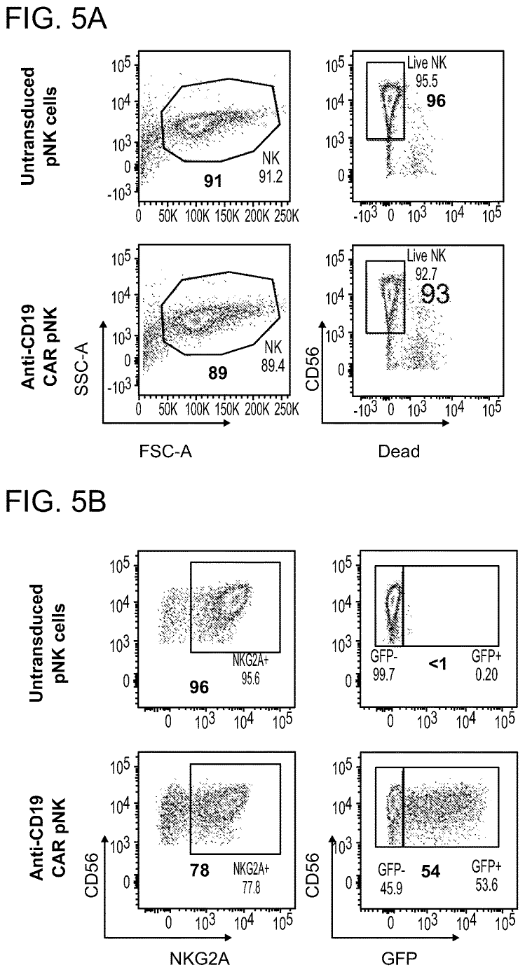

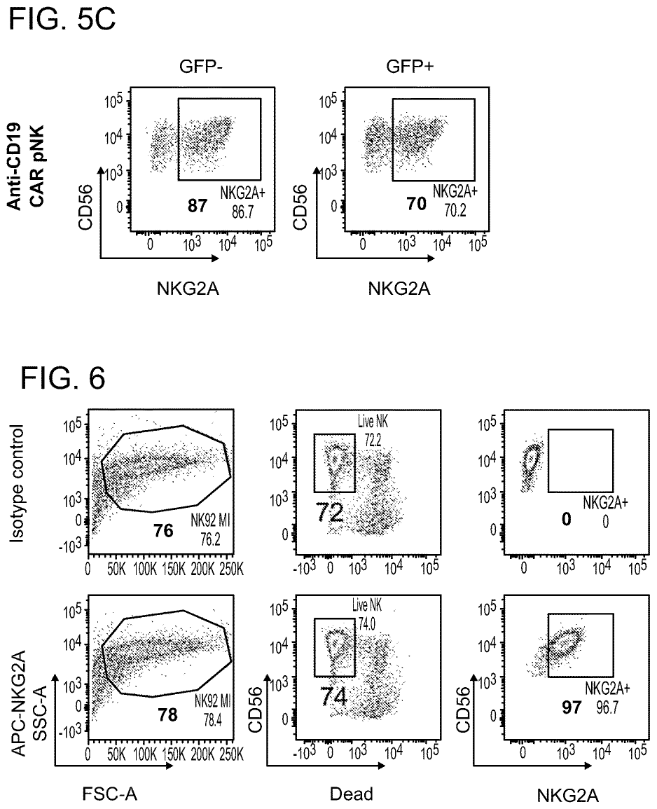

Expanded primary NK cells and NK cell lines also express NKG2A. After expansion, primary NK cells that were either untransduced (top panels) or transduced with a CAR-GFP construct (bottom panels) expressed CD56 (FIG. 5A-FIG. 5B). FIG. 5C shows cells gated on GFP and expressed both CD56 and NKG2A. Cells from the NK92 MI NK cell line also expressed NKG2A (FIG. 6); 97% of gated CD56⁺, live cells expressed NKG2A (FIG. 6, bottom right panel), whereas 0% of gated CD56⁺ cells were positive when stained with isotype control antibody (FIG. 6, top right panel).

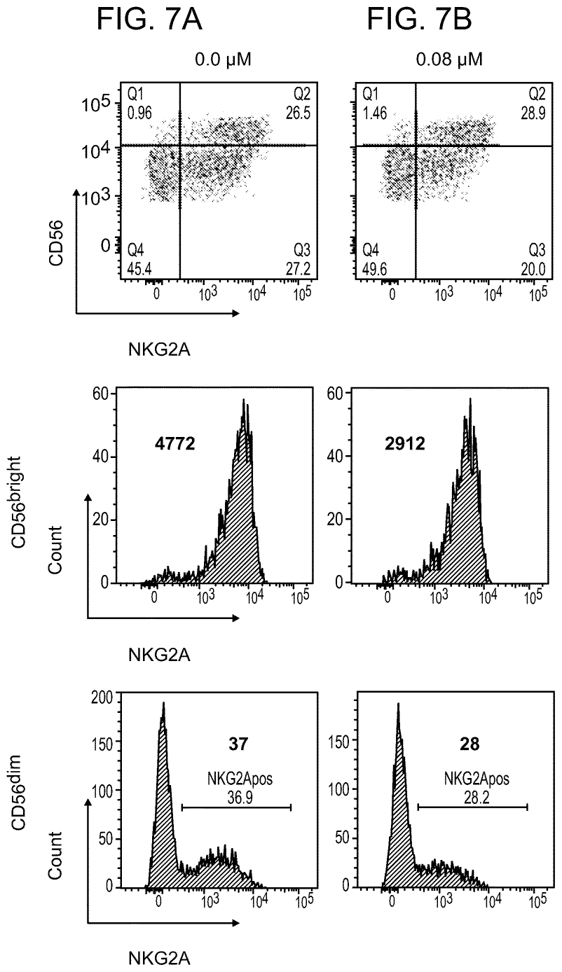

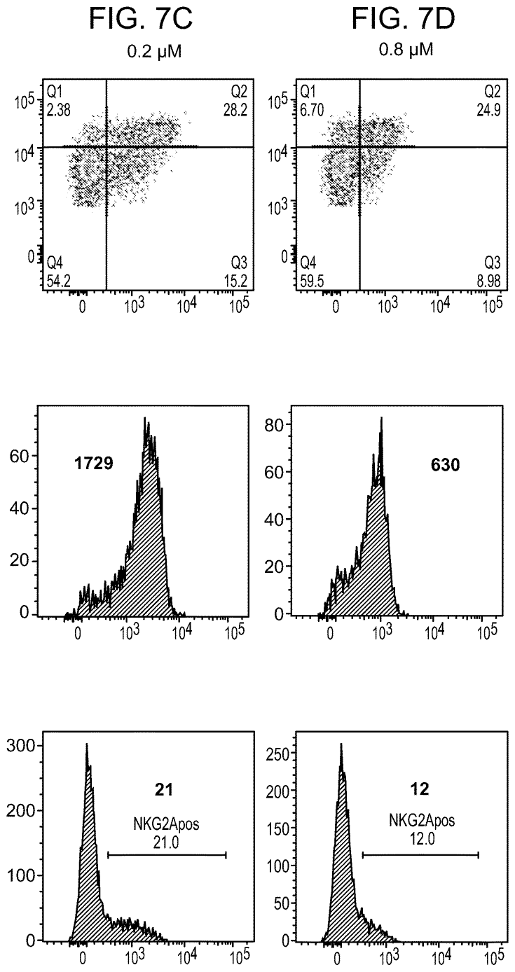

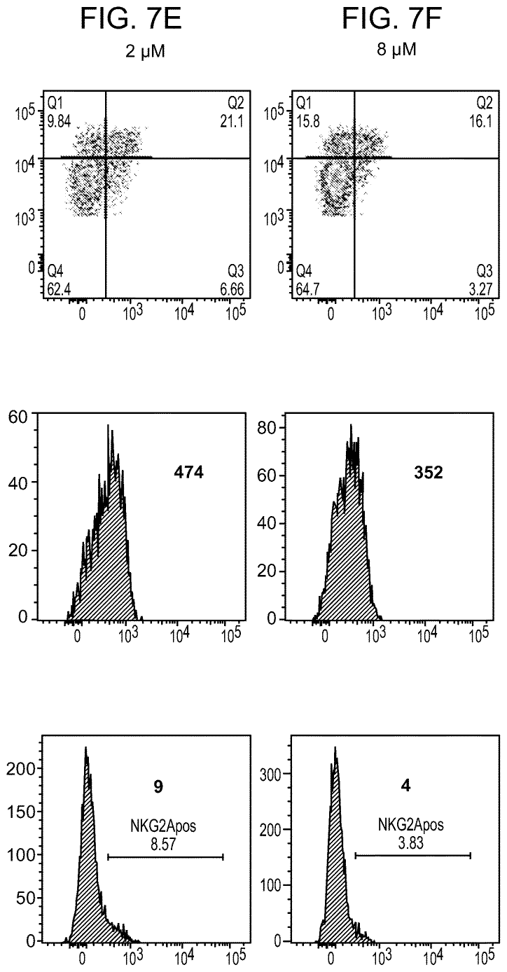

When challenged with an NKG2A-binding mabkine, anti-NKG2A antibodies did not bind to NKG2A+NK cells in a competitive binding assay. Primary NK cells were mixed with fluorescent anti-NKG2A antibodies without mabkines and the fluorescent anti-NKG2A antibody stained approximately 26.5% of the CD56⁺ cells when measured by flow cytometry (FIG. 7A). Increasing concentrations of NKG2A-binding mabkines of 0.08, 0.2, 0.8, 2 and 8 μM reduced fluorescent antibody binding to approximately 16% antibody stained CD56⁺ cells when the cells were challenged with 8 μM of mabkine (FIG. 7F).

### Example 4: NKG2A-Binding Mabkines Activate STAT5 in Primary NK Cells

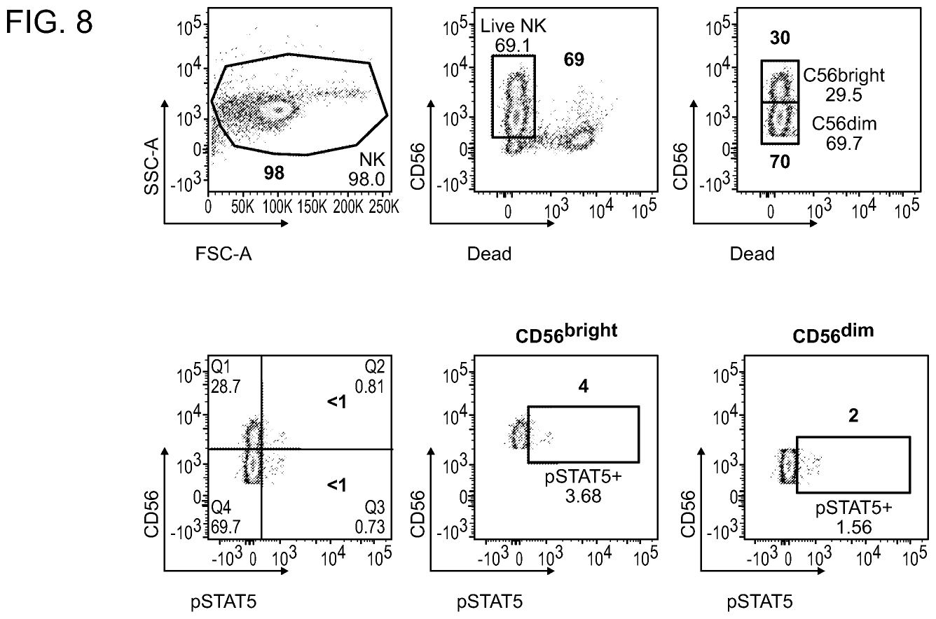

Signal transducer and activator of transcription (STAT) 5 is a master regulator of proliferation and lytic functions in NK cells. NKG2A-binding mabkines induce phosphorylation of STAT5, and therefore activation, of primary NK cells. FIG. 8 illustrates the flow cytometry gating strategy of purified NK cells into CD56^{bright} and CD56^{dim} NK cells and phosphorylated STAT5 (pSTAT5). Without stimulation, this gating strategy shows that very few of the gated CD56⁺ NK cells were positive for pSTAT5.

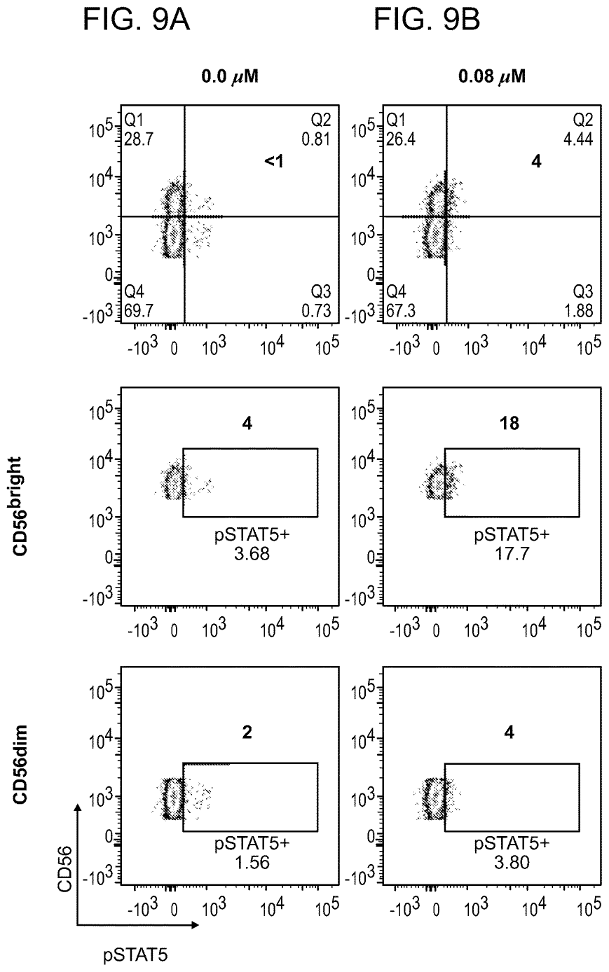

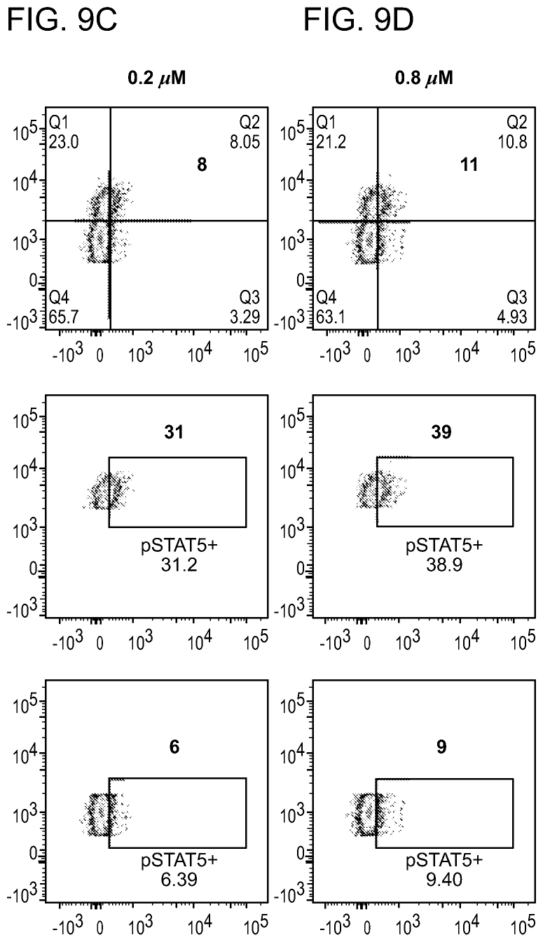

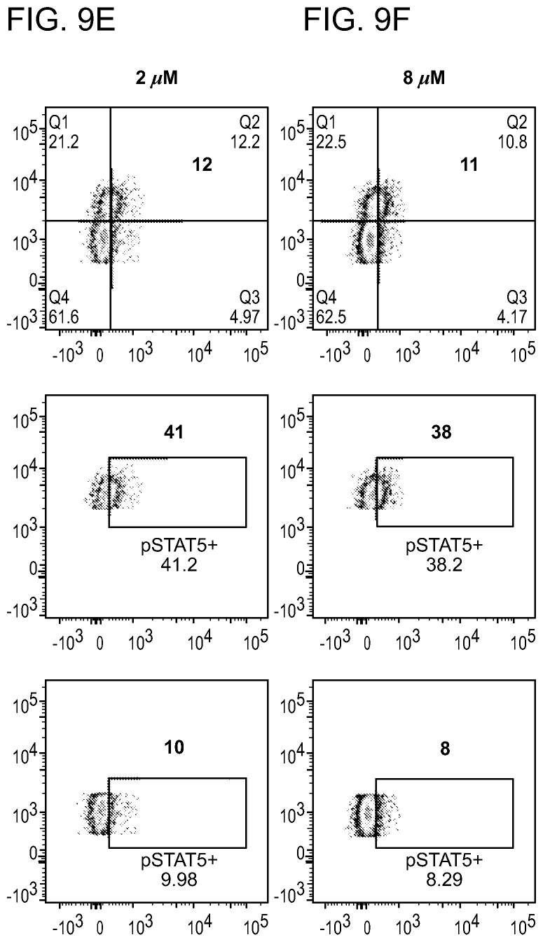

IL-15 was used as a positive control for NK cell activation. When challenged with an IL-15, CD56⁺ NK cells expressed pSTAT5 (FIG. 9A-FIG. 9F). As a negative control, primary NK cells were incubated without IL-15 and measured by flow cytometry for pSTAT5 staining (FIG. 9A). Without IL-15, only 4% of CD56^{bright} NK cells and 2% of CD56^{dim} NK cells were positive for pSTAT5. Increasing concentrations of IL-15 (0.08, 0.2, 0.8, 2 and 8 μM) increased pSTAT5 levels by about 10-fold. About 38% of CD56^{bright} NK cells were positive for pSTAT5 when stimulated with 8 μM IL-15, and only 8% of CD56^{dim} NK cells were positive for pSTAT5 when stimulated with 8 μM IL-15 (FIG. 9F).

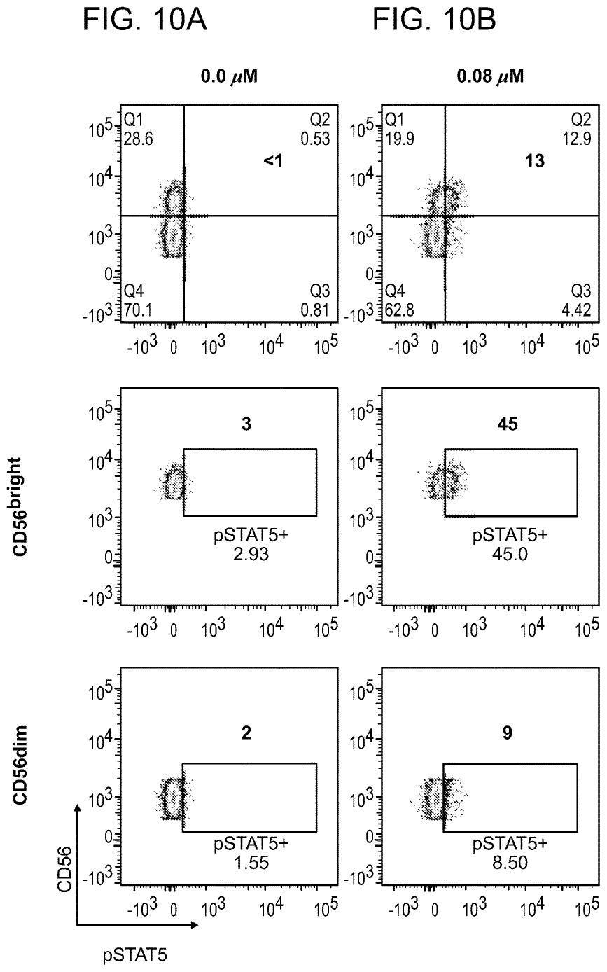

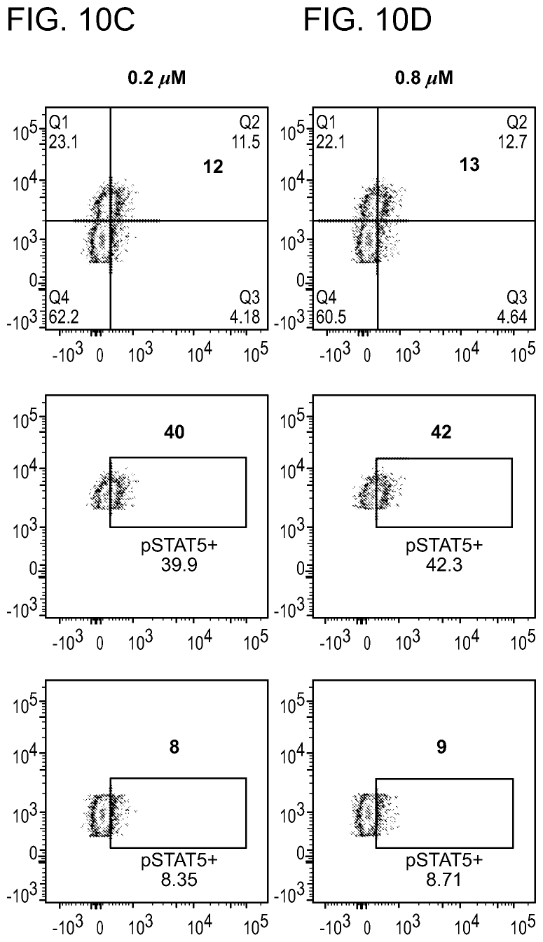

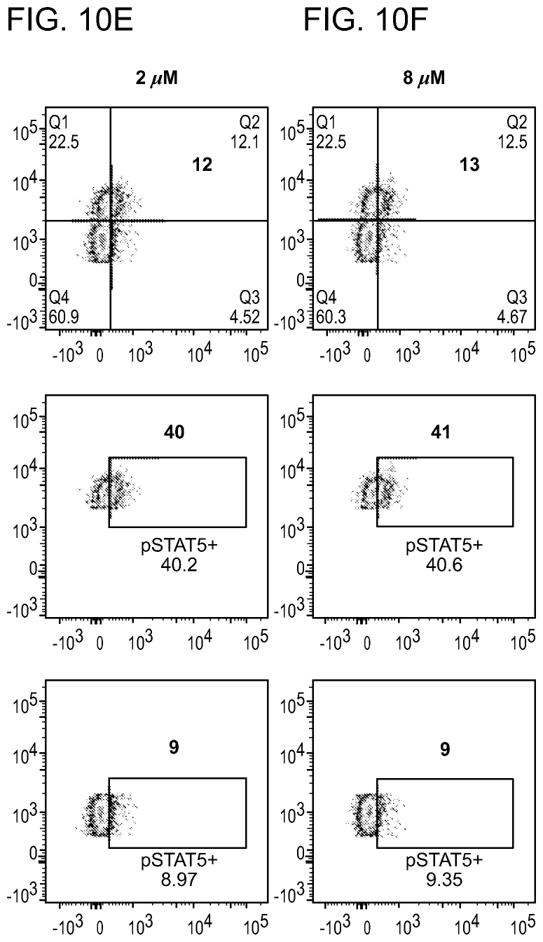

Next CD56⁺ NK cells were challenged with an NKG2A-binding mabkine and assayed for pSTAT5 expression (FIG. 10A-FIG. 10F). As a control, primary NK cells were incubated without mabkines and measured by flow cytometry for pSTAT5 staining (FIG. 10A). Without mabkines, only 3% of CD56^{bright} NK cells and 2% of CD56^{dim} NK cells were positive for pSTAT5. Increasing concentrations of NKG2A-binding mabkines (0.08, 0.2, 0.8, 2 and 8 μM) increased pSTAT5 levels by about 20-fold. About 41% of CD56^{bright} NK cells were positive for pSTAT5 when stimulated with 8 μM NKG2A-binding mabkines (FIG. 10F). Whereas only about 9% of CD56^{dim} NK cells were positive for pSTAT5 when stimulated with 8 μM NKG2A-binding mabkines (FIG. 10F). Furthermore, lower doses of NKG2A-binding mabkines (0.08 μM) also resulted in similar levels of pSTAT5 staining in both CD56^{bright} NK cells and CD56^{dim} NK cells, respectively (FIG. 10B-FIG. 10D).

### Example 5: NKG2A-Binding Mabkines Induce Primary NK Cell Killing

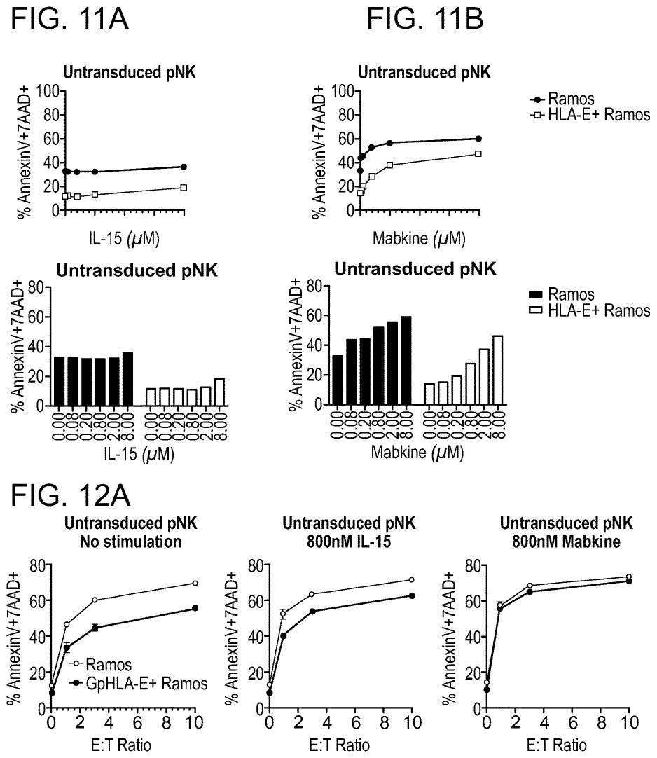

HLA-E is a ligand for the NK cell inhibitory receptor NKG2A (CD94) and is often overexpressed on cancer cells. The effect of NKG2A-binding mabkines on NK cell killing was determined by incubating primary NK cells with target cells with either IL-15 (positive control) or NKG2A-binding mabkines for 4 hours (FIG. 11A-FIG. 11B). IL-15 alone had minimal effects on NK cell killing of Ramos target cells or Ramos target cells expressing exogenous HLA-E (HLA-E+ Ramos) (FIG. 11A). NKG2A-binding mabkines induced NK cell killing of Ramos target cells (FIG. 11B) and to a lesser extent, HLA-E+ Ramos cells (FIG. 11B).

### Example 6: Mabkine Treatment Reverses HLA-E Induced Inhibition of Primary NK Cell Killing

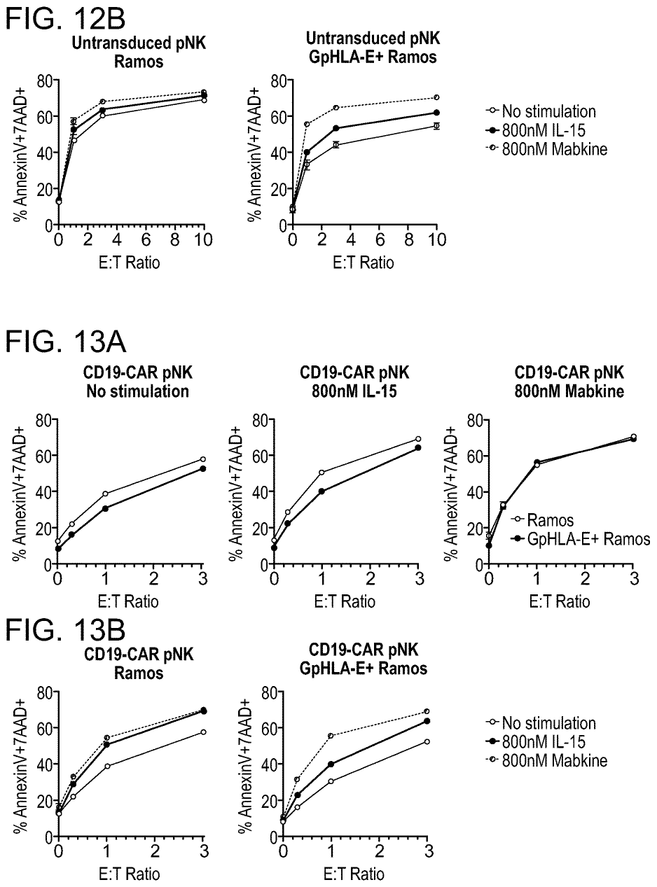

Primary NK (pNK) cells untransduced with any transgenes exhibited less target cell killing when incubated with Ramos target cells expressing HLA-E (GpHLA-E+ Ramos in FIG. 12A) as compared to Ramos cells not expressing HLA-E (labeled Ramos in FIG. 12A). Il-15 treatment enhanced NK cell killing of target cells expressing HLA-E; however, IL-15 treatment did not restore NK cell killing of HLA-E⁺ target cells as compared to HLA-E target cells (FIG. 12A, middle). Unlike IL-15 treatment, mabkine treatment enhanced and completely blocked the effect of HLA-E on NK cell killing, allowing NK cells to kill HLA-E⁺ target cells without apparent inhibition through HLA-E (FIG. 12A, right).

There were no major differences in NK cell killing of parental (HLA-E) Ramos cells between IL-15 or mabkine as compared to control (FIG. 12B, left). However, the effect of mabkine treatment was more potent than that of IL-15 in the increase NK cell killing of HLA-E′ target cells (FIG. 12B, right). This indicates that, in the absence of CAR expression, the mabkine helped NK cells overcome the inhibition through HLA-E and enhanced NK cell killing of the target cells.

pNK cells were transduced with a CAR that binds CD19 and treated with IL-15 or mabkine to investigate the synergistic effect of mabkine and CAR expression on immune cell killing (FIG. 13A). CAR-NK cells had a slight decrease in killing of HLA-E⁺ Ramos cells as compared to HLA-E Ramos cells, indicating that HLA-E also effects CAR-NK cells (FIG. 13A, left) killing. IL-15 stimulation had little to no effect on CAR-NK cell killing of HLA-E⁺ target cells as compared to killing of HLA-E⁻ target cells (FIG. 12A, middle). Mabkine stimulation completely blocked the inhibitory effect of HLA-E on CAR-NK cell killing and allowed the CAR-NK cells to kill the HLA-E⁺ target cells without apparent inhibition through HLA-E.

Similar to the untransduced pNK cell killing, there were no major differences in CAR-NK cell killing of parental (HLA-E⁻) Ramos cells between IL-15 or mabkine as compared to control (FIG. 13B, left). The mabkine treatment was more effective than that of IL-15 treatment in the increased CAR-NK cell killing of HLA-E⁺ target cells (FIG. 13B, right). This data shows that although CAR-NK cells are very effective at killing target cells expressing HLA-E, the mabkine further enhanced NK cell killing of HLA-E⁺ target cells.

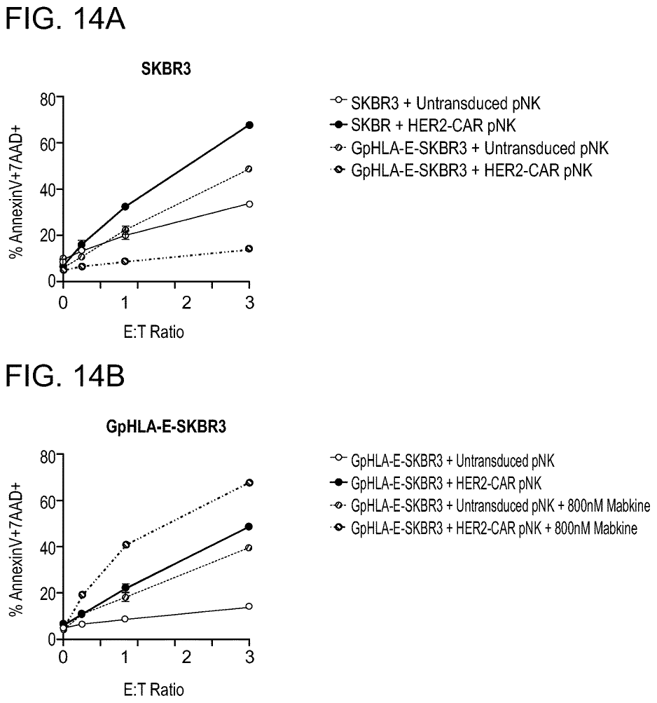

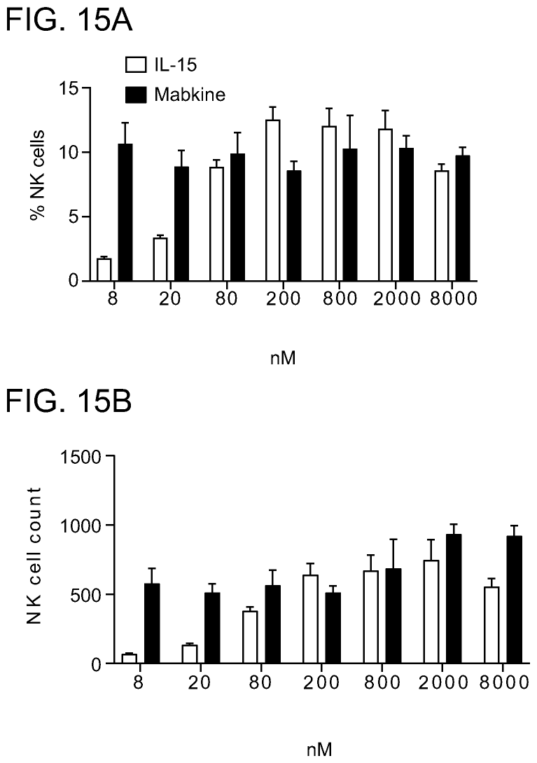

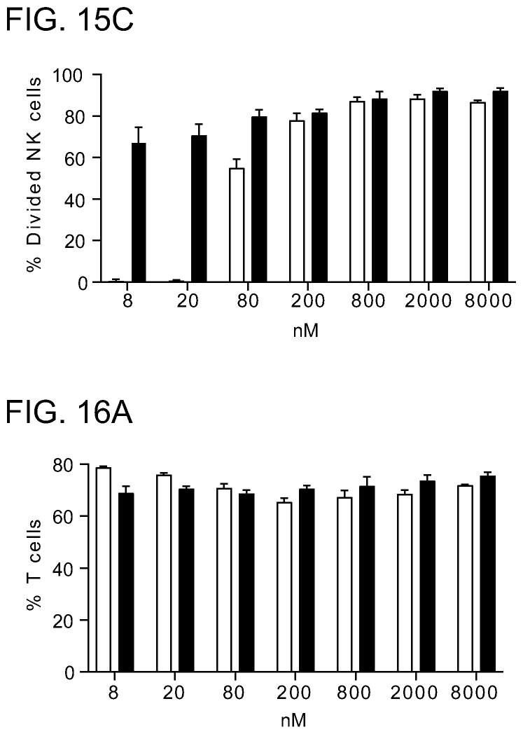

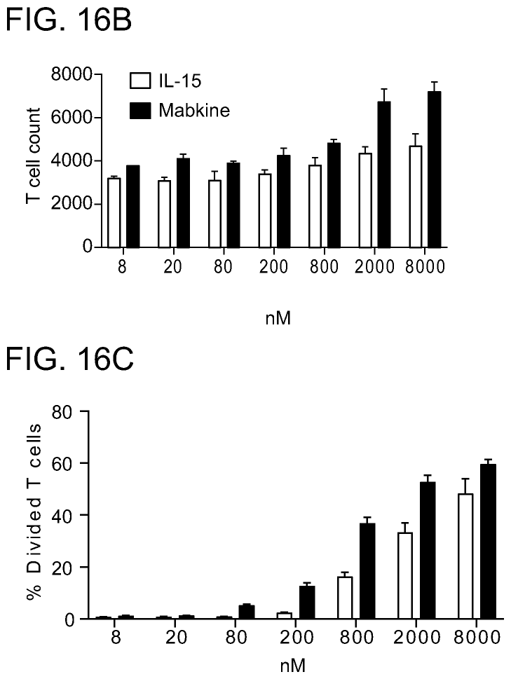

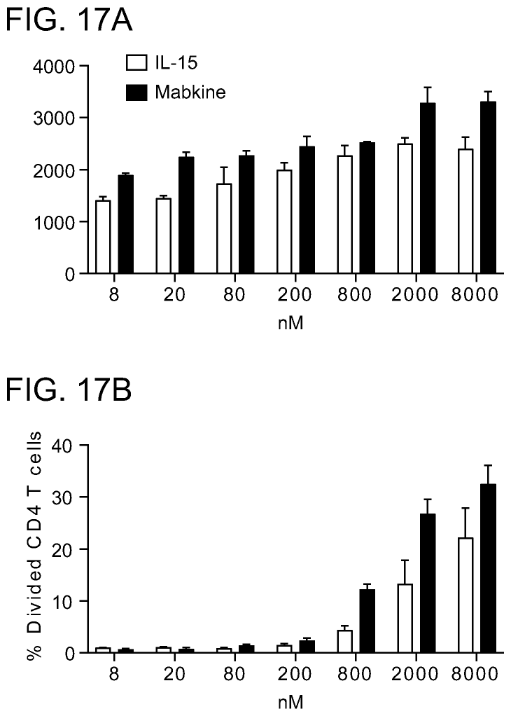

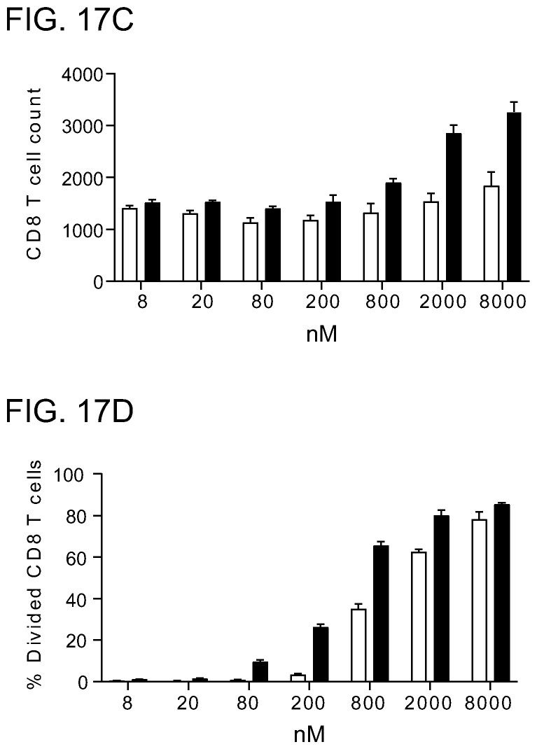

The effect mabkine on NK cell killing of the human breast cancer cell line SKBR3 was next investigated. SKBR3 cells overexpresses HER2 (Neu/ErbB-2). Both untransduced pNK cells and NK cells expressing a CAR that binds HER2 (HER2-CAR pNK) had decreased killing of HLA-E⁺ SKBR3 cells, indicating that HLA-E inhibits NK cell killing of target cells even in when the NK cell expresses a CAR. Despite this reduced killing, HER2-CAR pNK cells were much more effective than untransduced pNK cells in killing both HLA-E⁻ parental SKBR3 cells and HLA-E⁺ SKBR3 cells (FIG. 14A).

Mabkine treatment further enhanced the ability of both untransduced pNK cells and HER2-CAR pNK cells to kill of HLA-E⁺ SKBR3 cells (FIG. 14B). This data shows that combining a CAR-expressing cell with mabkine treatment leads to robust NK cell killing of target cells expressing HLA-E and demonstrates utility of using mabkines in CAR immune cell therapy in cancer patients.

### Example 7: Mabkine Stimulation Results in PBMC Proliferation

Peripheral blood mononuclear cells (PBMCs) were isolated from a leukoreduction system chamber from a healthy donor using Ficoll® density gradient centrifugation. The PBMCs were then labelled with CellTrace™ Cell Proliferation Kit (Invitrogen) to track cellular division, then stimulated with different concentrations of recombinant human IL-15 (Miltenyi) or mabkine for a total of 7 days before being assessed by flow cytometry. Cells were stained with Biolegend antibodies anti-CD56 (clone HCD56), anti-CD16 (clone 3G8), anti-CD3 (clone OKT3), anti-CD4 (clone RPA-T4), and anti-CD8 (clone SK1). Fixable Yellow Live/Dead stain from Invitrogen was added during staining to exclude dead cells. Cells were acquired using a BD LSRFortessa™ and analyzed using FlowJo™ (Tree Star). Graphs were generated using GraphPad Prism™ 10 software.

The mabkine induced NK cell proliferation, even at very low concentrations (as low as 8 nM) and was more effective at inducing NK cell proliferation than the same concentration of IL-15. The mabkine induced T cell proliferation at 80 nM and was more effective than the same concentration of IL-15. The mabkine induced CD8⁺ T cell proliferation at 80 nM and CD4⁺ T proliferation at 800 nM and was more effective at both CD8⁺ and CD4⁺ T cell proliferation than the same concentration of IL-15.

Therefore, low concentrations of the mabkine were sufficient to induce robust proliferation of both NK cells and T cells. Without being bound by theory, the mabkine could be very effective in vivo at low doses, potentially translating to reduced levels of in vivo toxicity.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A fusion protein (mabkine), comprising:
a target binding domain that binds an antigen on a cancer cell, a cognate receptor of the antigen, or a cognate ligand of the antigen, wherein the cognate receptor and the cognate ligand are present on an immune cell;
a hinge domain;
an IL-15Rα domain;
a linker; and
an IL-15 domain;
wherein the hinge domain is disposed between the target binding domain and the IL-15Rα domain, and the linker is disposed between the IL-15Rα domain and the IL-15 domain.

2. The mabkine of claim 1, wherein the target binding domain binds NKG2A, HLA-E, EGFR, or PD-L1.

3. The mabkine of claim 2, wherein the target binding domain binds NKG2A.

4. The mabkine of claim 3, wherein the target binding domain comprises a single chain fragment of an antibody that binds NKG2A.

5. The mabkine of claim 4, wherein the single chain fragment is a single chain variable fragment of Monalizumab.

6. The mabkine of claim 4, wherein the single chain fragment comprises a variable light (VL) domain and a variable heavy (VH) domain.

7. The mabkine of claim 6, wherein the VL domain comprises the amino acid sequence of SEQ ID NO: 17 and the VH domain comprises the amino acid sequence of SEQ ID NO: 18.

8. The mabkine of claim 2, wherein the target binding domain binds HLA-E.

9. The mabkine of claim 8, wherein the target binding domain comprises a single chain fragment of an antibody that binds HLA-E.

10. The mabkine of claim 2, wherein the target binding domain binds EGFR.

11. The mabkine of claim 10, wherein the target binding domain comprises a single chain fragment of an antibody that binds EGFR.

12. The mabkine of claim 11, wherein the single chain fragment is a single chain variable fragment of Cetuximab.

13. The mabkine of claim 2, wherein the target binding domain binds PD-1.

14. The mabkine of claim 13, wherein the target binding domain comprises a single chain fragment of an antibody that binds PD-1.

15. The mabkine of claim 14, wherein the single chain fragment is a single chain variable fragment of Balstilimab, Nivolumab, or Pembrolizumab.

16. The mabkine of claim 2, wherein the target binding domain binds PD-L1.

17. The mabkine of claim 16, wherein the target binding domain comprises a single chain fragment of an antibody that binds PD-L1.

18. The mabkine of claim 17, wherein the single chain fragment is a single chain variable fragment of Durvalumab.

19. The mabkine of any one of claims 1-18, wherein the IL-15 domain comprises full-length IL-15, mature IL-15, or N72D mutated IL-15

20. The mabkine of claim 19, wherein the IL-15 domain has an amino acid sequence
(SEQ ID NO: 31)
NWVNVISDLKKIEDLIQSMHIDATLYTESDVHPSCKVTAMKCFLL
ELQVISLESGDASIHDTVENLIILANNSLSSNGNVTESGCKECEE
LEEKNIKEFLQSFVHIVQMFINTS.

21. The mabkine of any one of claims 1-20, wherein the IL-15Rα domain has the amino acid sequence
(SEQ ID NO: 28)
ITCPPPMSVEHADIWVKSYSLYSRERYICNSGFKRKAGTSSLTEC
VLNKATNVAHWTTPSLKCIR.

22. The mabkine of any one of claims 1-21, wherein the linker comprises the amino acid sequence GGGGS (SED ID NO: 34).

23. The mabkine of claim 22, wherein the linker comprises the amino acid sequence
(SEQ ID NO: 38)
SGGSGGGGSGGGSGGGGSLQ.

24. The mabkine of any one of claims 1-23, wherein the hinge domain is derived from IgG1.

25. The mabkine of claim 24, wherein the hinge domain comprises the amino acid sequence
(SEQ ID NO: 23)
DKTHTCPPCPAPELLGGP.

26. The mabkine of any one of claims 1-25, wherein the hinge domain is derived from IgG4.

27. The mabkine of claim 26, wherein the hinge domain comprises the amino acid sequence
(SEQ ID NO: 25)
PPCPPCPAPEFLGGP
or
(SEQ ID NO: 26)
PPCPSCPAPEFLGGP

28. The mabkine of claim 27, wherein the hinge domain comprises the amino acid sequence of SEQ ID NO: 59.

29. The mabkine of any one of claims 1-28, further comprising a signal peptide derived from IL-2, wherein the signal peptide is disposed N-terminal to the target binding domain.

30. The mabkine of any one of claims 1-29, further comprising an Fc domain disposed between the hinge domain and the IL-15Rα domain.

31. The mabkine of claim 30, wherein the Fc domain is not derived from IgG1.

32. The mabkine of claim 30 or 31, wherein the Fc domain is derived from IgG4.

33. The mabkine of claim 33, wherein the Fc domain has the amino acid sequence
(SEQ ID NO: 43)
SVFLFPPKPKDTLMISRTPEVTCVVVDVSQEDPEVQFNWYVDGVE
VHNAKTKPREEQFNSTYRVVSVLTVLHQDWLNGKEYKCKVSNKGL
PSSIEKTISKAKGQPREPQVYTLPPSQEEMTKNQVSLTCLVKGFY
PSDIAVEWESNGQPENNYKTTPPVLDSDGSFFLYSRLTVDKSRWQ
EGNVFSCSVMHEALHNHYTQKSLSLSLGK.

34. The mabkine of any one of claims 1-33, in the form of a homodimer comprising two of the fusion proteins.

35. A nucleic acid that encodes the mabkine of any one of claims 1-33.

36. An expression vector comprising the nucleic acid of claim 35.

37. The expression vector of claim 36, which is a viral vector.

38. The expression vector of claim 36, which is a non-viral vector.

39. The expression vector of claim 38, which is a plasmid vector.

40. The expression vector of any one of claims 36-39, wherein the nucleic acid has the sequence of SEQ ID NOs: 68-70.

41. A genetically modified cell comprising the expression vector of any one of claims 36-40.

42. The genetically modified cell of claim 41, which is a CHO-S, a HEK293, PER.C6, NS0, Sp2/0 cell line or derivative thereof.

43. The genetically modified cell of claim 41, which is a mammalian immune cell.

44. The genetically modified cell of claim 43 (CAR-immune cell), further comprising a nucleic acid that encodes a chimeric antigen receptor (CAR) that comprises an extracellular domain that binds an antigen present on a cancer cell, a transmembrane domain, and an intracellular domain comprising a stimulatory domain.

45. A pharmaceutical composition comprising the mabkine of any one of claims 1-34, and a pharmaceutically acceptable carrier.

46. A pharmaceutical composition, comprising an effective number of the genetically modified immune cells of claim 43, and a pharmaceutically acceptable carrier.

47. A pharmaceutical composition, comprising an effective number of the genetically modified immune cells of claim 44, and a pharmaceutically acceptable carrier.

48. A method of making a mabkine comprising:
introducing the expression vector of any one of claims 36-40 into a cell; and
culturing the cell in a medium under conditions wherein the nucleic acid encoding the mabkine is expressed; and
isolating the mabkine from the cell and/or medium.

49. The method of claim 48, wherein the mabkine is isolated by binding the mabkine to an affinity chromatography resin in a neutral phosphate buffer.

50. The method of claim 49, further comprising subjecting the affinity chromatography resin with an acidic buffer with a pH of about 3 to about 4.

51. The method of claim 50, further comprising:
neutralizing the acidic buffer; and
performing a tangential flow filtration

52. The method of any one of claims 49-51, wherein the affinity chromatography resin is a Protein A affinity chromatography resin or a Protein G affinity chromatography resin.

53. A method of treating cancer, comprising:
administering, to a subject in need thereof, an effective amount of the mabkine of any one of claims 1-34, or the pharmaceutical composition of any one of claims 45-47.

54. The method of claim 53, wherein the cancer is a hematological cancer.

55. The method of claim 54, wherein the hematological cancer is multiple myeloma, lymphoma, or leukemia.

56. The method of claim 53, wherein the cancer is characterized by a solid tumor.

57. The method of claim 56, wherein the cancer is bladder cancer, renal cell carcinoma, melanoma, or non-small cell lung cancer (NSCLC), squamous cell carcinoma of the head and neck (SCCHN) and colorectal carcinoma (CRC), ovarian cancer, endometrial cancer, cervical cancer, kidney renal clear cell carcinoma (KIRC), head and neck squamous cell carcinoma (HNSC), lung adenocarcinoma (LUAD), skin cutaneous melanoma (SKCM), prostate adenocarcinoma (PRAD), stomach adenocarcinoma (STAD), colon adenocarcinoma (COAD), cervical squamous cell carcinoma and endocervical adenocarcinoma (CESC), lung squamous cell carcinoma (LUSC), or rectum adenocarcinoma (READ).

58. The method of claim 53, wherein the cancer is leukemia or head and neck cancer.

59. The method of any one of claims 53-58, wherein the mabkine or the pharmaceutical composition is administered parenterally.

60. The method of any one of claims 53-57, wherein the mabkine or the pharmaceutical composition is administered intratumorally.

61. The method of any one of claims 53-60, comprising administering to the subject in need thereof, an effective amount of the mabkine of any one of claims 1-34, or the pharmaceutical composition of any one of claims 45-46, and wherein the method further comprises administering to the subject a CAR-immune cell.

62. The method of claim 61, wherein the immune cell is a NK cell.

63. The method of claim 61, wherein the immune cell is a T cell.

64. The method of claim 63, wherein the immune cell further comprises a nucleic acid encoding a chimeric antigen receptor (CAR) comprising an antigen binding domain that binds an antigen present on a cancer cell of the subject, a transmembrane domain, and an intracellular domain comprising a stimulatory domain.

65. The method of any one of claims 61-64, wherein the immune cell is administered prior to the administration of the mabkine or the pharmaceutical composition.

66. The method of any one of claims 53-65, further comprising administering a PD1 inhibitor.

67. The method of claim 66, wherein the PD1 inhibitor comprises durvalumab, nivolumab, or pembroliumab.


SEQUENCE LISTING (87 sequences)

SEQ ID NO: 1 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 221 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 4 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 11 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 452 aa, synthetic construct
EVQLVQSGAEVKKPGESLKISCKGSGYSFTSYWMNWVRQMPGKGLEWMGRIDPYDSETHYSPSFQGQVTISADKSISTAYLQWSSLKASDTAMYYCARGGYDFDVGTLYWFFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 214 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 16 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 17 - protein, 107 aa, synthetic construct
DIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIK

SEQ ID NO: 18 - protein, 125 aa, synthetic construct
QVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSS

SEQ ID NO: 19 - protein, 9 aa, Homo sapiens
QSFGLLDPK

SEQ ID NO: 20 - protein, 23 aa, Homo sapiens
LSEGDKVKMDSRIQVLSRGVNQT

SEQ ID NO: 21 - protein, 49 aa, Homo sapiens
KPTTTPAPRPPTPAPTIASQPLSKRPEACRPAAGGAVHTRGLDFACDIY

SEQ ID NO: 22 - protein, 39 aa, Homo sapiens
IEVMYPPPYLDNERSNGTIIHVKGKHLCPSPLFPGPSKP

SEQ ID NO: 23 - protein, 18 aa, Homo sapiens
DKTHTCPPCPAPELLGGP

SEQ ID NO: 24 - protein, 18 aa, Homo sapiens
ERKCCVECPPCPAPPAAA

SEQ ID NO: 25 - protein, 15 aa, Homo sapiens
PPCPPCPAPEFLGGP

SEQ ID NO: 26 - protein, 15 aa, Homo sapiens
PPCPSCPAPEFLGGP

SEQ ID NO: 27 - protein, 353 aa, Homo sapiens
MRLAGRQVPEQRSPPPPGLGSARPGSPAVSCGAAAMAPRRARGCRTLGLPALLLLLLLRPPATRDARDRLAVLAGRSRISESFNHEVQTHEACVRLRTMENCPQCHHHRTSRQQAGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 28 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 29 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 5 aa, synthetic construct
GGGGX

SEQ ID NO: 33 - protein, 6 aa, synthetic construct
GSSGSX

SEQ ID NO: 34 - protein, 5 aa, synthetic construct
GGGGS

SEQ ID NO: 35 - protein, 10 aa, synthetic construct
GGGGSGGGGS

SEQ ID NO: 36 - protein, 15 aa, synthetic construct
GGGGSGGGGSGGGGS

SEQ ID NO: 37 - protein, 20 aa, synthetic construct
GGGGSGGGGSGGGGSGGGGS

SEQ ID NO: 38 - protein, 20 aa, synthetic construct
SGGSGGGGSGGGSGGGGSLQ

SEQ ID NO: 39 - protein, 18 aa, synthetic construct
GSTSGSGKPGSGEGSTKG

SEQ ID NO: 40 - protein, 18 aa, synthetic construct
KESGSVSSEQLAQFRSLD

SEQ ID NO: 41 - protein, 14 aa, synthetic construct
EGKSSGSGSESKST

SEQ ID NO: 42 - protein, 12 aa, synthetic construct
GSAGSAAGSGEF

SEQ ID NO: 43 - protein, 209 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 18 aa, Homo sapiens
MKWVTFISLLFLFSSAYS

SEQ ID NO: 45 - protein, 19 aa, synthetic construct
MKWVTFISLLFLFSSSSRA

SEQ ID NO: 46 - protein, 21 aa, Homo sapiens
MALPVTALLLPLALLLHAARP

SEQ ID NO: 47 - protein, 16 aa, Homo sapiens
MPLLLLLPLLWAGALA

SEQ ID NO: 48 - protein, 27 aa, Homo sapiens
MGVHECPAWLWLLLSLLSLPLGLPVLG

SEQ ID NO: 49 - protein, 20 aa, Homo sapiens
MYRMQLLSCIALSLALVTNS

SEQ ID NO: 50 - protein, 20 aa, Mus musculus
METDTLLLWVLLLWVPGSTG

SEQ ID NO: 51 - protein, 20 aa, Homo sapiens
MEAPAQLLFLLLLWLPDTTG

SEQ ID NO: 52 - protein, 20 aa, synthetic construct
MEAPAQLLFLLLLWLPSSRA

SEQ ID NO: 53 - protein, 23 aa, Homo sapiens
MDAMKRGLCCVLLLCGAVFVSPS

SEQ ID NO: 54 - protein, 17 aa, Homo sapiens
MLLLLLLLGLRLQLSLG

SEQ ID NO: 55 - protein, 16 aa, synthetic construct
MLLLLLLLLLLALALA

SEQ ID NO: 56 - protein, 21 aa, synthetic construct
MWWRLWWLLLLLLLLWPMVWA

SEQ ID NO: 57 - protein, 6 aa, synthetic construct
DTYRYI

SEQ ID NO: 58 - protein, 6 aa, synthetic construct
TDFYLK

SEQ ID NO: 59 - protein, 6 aa, synthetic construct
HHHHHH

SEQ ID NO: 60 - protein, 7 aa, synthetic construct
DYKDDDK

SEQ ID NO: 61 - protein, 24 aa, synthetic construct
EFDYKDDDDKGSLPETGGHHHHHH

SEQ ID NO: 62 - protein, 19 aa, synthetic construct
KDHLIHNVHKEFHAHAHNK

SEQ ID NO: 63 - protein, 8 aa, synthetic construct
QPELAPED

SEQ ID NO: 64 - protein, 690 aa, synthetic construct
MYRMQLLSCIALSLALVTNSDIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIKGGGGSGGGGSGGGGSQVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 65 - protein, 699 aa, synthetic construct
MYRMQLLSCIALSLALVTNSHHHHHHVVHDIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIKGGGGSGGGGSGGGGSQVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 493 aa, synthetic construct
MYRMQLLSCIALSLALVTNSHHHHHHVVHDIQMTQSPASLSASVGETVTITCRASENIYSYLAWYQQKQGKSPQFLVYNAKTLAEGVPSRFSGSGSGTQFSLKINSLQPEDFGSYYCQHHYGTPRTFGGGTKLEIKGGGGSGGGGSGGGGSQVQLQQPGAELVRPGASVKLSCKASGYTFTSYWMNWVKQRPEQGLQWIGRIDPYDSETHYSQKFKDKAILTVDKSSSTAYMRLSSLTSEDSAVYYCARGGYDFDVGTLYWFFDVWGAGTTVTVSSDKTHTCPPCPAPELLGGPITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - DNA, 81 bp, synthetic construct
  source 1..81: mol_type = other DNA
gctagcgctaccggactcagatctcgagctcaagcttcgaattctgcagtcgacggtaccgcgggcccgggatccgccacc

SEQ ID NO: 68 - DNA, 2073 bp, synthetic construct
  source 1..2073: mol_type = other DNA
atgtacaggatgcaactcctgtcttgcattgcactaagtcttgcacttgtcacgaattcggacatccagatgactcagtctccagcctccctatctgcatctgtgggagaaactgtcaccatcacgtgtcgagcaagtgagaatatttacagttatttagcatggtatcagcagaaacagggaaaatctcctcagttcttggtctataatgcaaaaaccttagcagaaggtgtgccatcaaggttcagtggcagtggatcaggcacacagttttctctgaagatcaacagcctacagcctgaagattttgggagttattactgtcaacatcactatggtactcctcggacgttcggtggaggcaccaagctggaaatcaaaggtggaggtggcagcggaggaggtgggtccggcggtggaggaagccaggtccaactgcagcagcctggggctgagctggtgaggcctggggcttcagtgaagctgtcctgcaaggcttctggctacacgttcaccagctactggatgaactgggttaagcagaggcctgagcaaggccttcagtggattggaaggattgatccttacgatagtgaaactcactacagtcaaaagttcaaggacaaggccatattgactgtagacaaatcctccagcacagcctacatgcgactcagcagcctgacatctgaggactctgcggtctattactgtgcaagagggggctatgatttcgacgtaggaactctctactggttcttcgatgtctggggcgcagggaccacggtcaccgtctcctcacccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagaagcggcggcagcggcggcggcggcagcggcggcggcagcggcggcggcggcagcctgcagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttaa

SEQ ID NO: 69 - DNA, 2088 bp, synthetic construct
  source 1..2088: mol_type = other DNA
atgtacaggatgcaactcctgtcttgcattgcactaagtcttgcacttgtcacgaattcgcatcaccatcaccatcacgacatccagatgactcagtctccagcctccctatctgcatctgtgggagaaactgtcaccatcacatgtcgagcaagtgagaatatttacagttatttagcatggtatcagcagaaacagggaaaatctcctcagttcttggtctataatgcaaaaaccttagcagaaggtgtgccatcaaggttcagtggcagtggatcaggcacacagttttctctgaagatcaacagcctacagcctgaagattttgggagttattactgtcaacatcactatggtactcctcggacgttcggtggaggcaccaagctggaaatcaaaggtggaggtggcagcggaggaggtgggtccggcggtggaggaagccaggtccaactgcagcagcctggggctgagctggtgaggcctggggcttcagtgaagctgtcctgcaaggcttctggctacacgttcaccagctactggatgaactgggttaagcagaggcctgagcaaggccttcagtggattggaaggattgatccttacgatagtgaaactcactacagtcaaaagttcaaggacaaggccatattgactgtagacaaatcctccagcacagcctacatgcgactcagcagcctgacatctgaggactctgcggtctattactgtgcaagagggggctatgatttcgacgtaggaactctctactggttcttcgatgtctggggcgcagggaccacggtcaccgtctcctcacccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagaagcggcggcagcggcggcggcggcagcggcggcggcagcggcggcggcggcagcctgcagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 70 - DNA, 2088 bp, synthetic construct; identical to SEQ ID NO: 69
  source 1..2088: mol_type = other DNA

SEQ ID NO: 71 - protein, 21 aa, Homo sapiens
LCYLLDGILFIYGVILTALFL

SEQ ID NO: 72 - protein, 22 aa, Homo sapiens
MALIVLGGVAGLLLFIGLGIFF

SEQ ID NO: 73 - protein, 24 aa, Homo sapiens
IYIWAPLAGTCGVLLLSLVITLYC

SEQ ID NO: 74 - protein, 27 aa, Homo sapiens
FWVLVVVGGVLACYSLLVTVAFIIFWV

SEQ ID NO: 75 - protein, 27 aa, Homo sapiens
IISFFLALTSTALLFLLFFLTLRFSVV

SEQ ID NO: 76 - protein, 440 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 448 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 214 aa, synthetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC